(19) United States
(12) Patent Application Publication
(10) Pub. No.: US 2025/0170317 A1
(43) Pub. Date: May 29, 2025

(54) SICKLE CELL DISEASE TREATMENT SYSTEM AND METHOD

(71) Applicant: Michael Tarasev, Ysilanti, MI (US)

(72) Inventor: Michael Tarasev, Ysilanti, MI (US)

(21) Appl. No.: 18/815,600

(22) Filed: Aug. 26, 2024

Related U.S. Application Data
(60) Provisional application No. 63/535,377, filed on Aug. 30, 2023.

(51) Int. Cl. A61M 1/36

(52) U.S. Cl. CPC A61M 1/3603; A61M 1/3621; A61M 2202/0007; A61M 2202/0014; A61M 2202/0021; A61M 2202/0028; A61M 2202/0042; A61M 2202/005; A61M 2202/0064; A61M 2202/0078; A61M 2202/0208; A61M 2202/0413; A61M 2205/7545

(57) ABSTRACT

A method for treating sickle cell disease comprises removing blood from a patient with sickle cell disease, treating the removed blood to decrease the fraction of sickle-prone cells in the removed blood, and reintroducing the treated blood back into the patient. The treated blood that is reintroduced back into the patient has a reduced number of sickle-prone red blood cells when compared to the blood removed from the patient. In one version, the treatment of the blood includes inducing sickling of the red blood cells prone to sickle and then separating those sickled red blood cells from the blood before reintroducing the blood back into the patient.

### PRIORITY

The present application claims the benefit of domestic priority based on U.S. Provisional Patent Application 63/535,377 filed on Aug. 30, 2023, the entirety of which is incorporated herein by reference.

### BACKGROUND

Sickle Cell Disease is an inherited red blood cell disorder that affects up to 100,000 people in the United States and 20 million people worldwide. People with this disease have atypical hemoglobin molecules often called hemoglobin S (sickle hemoglobin, often abbreviated HbS or Hb-S).

Sickle cell disease causes red blood cells that are normally disc-shaped and flexible to change shape, such as by becoming sickle-shaped or other shape and/or becoming less flexible. The sickled red blood cells can block blood flow to parts of the body which can lead to serious problems. For example, complications and symptoms from sickle cell disease can include chronic anemia, pain crises, vaso-occlusive crisis, stroke, acute chest syndrome, pulmonary hypertension, splenic and renal dysfunction. Also, due to extreme membrane damage, sickled red blood cells survive only about 20 days as opposed to the 120 day life of healthy red blood cells.

There are presently few effective treatment options for sickle cell disease. Conventional courses of treatment involve pain management, treatment with oral drug hydroxyurea, and/or red blood cell transfusion therapy. Treatment with the drug Hydroxyurea remains the most commonly used drug treatment. It was shown to induce hemoglobin F (fetal hemoglobin, often appreviated as HbF or Hb-F) production leading to development of red blood cells containing in addition to hemoglobin S, which is responsible for red blood cell sickling, also variable amounts of hemoglobin F. The more hemoglobin F is contained in such red blood cells, the less likely are such cells to sickle. The amount of hemoglobin F generated due to the treatment and its distribution among the red blood cells produced was shown to be highly variable and patient-dependent. Recently, several drugs have been introduced to help managing sickle cell disease. Adakveo (crizanlizumab) reduces red and white cell binding to blood vessel wall thus reducing the risks of development of vaso-occlusion. Endari (L-Glutamine) improves energy production in red blood cells, which supports better anti-oxidation activity resulting, it is believed, in reduced oxidative damage to red blood cell membrane. Oxbryta (voxelotor) binds to hemoglobin molecule leading to delayed red blood cell sickling. A number of other drugs with similar mechanisms of action are currently in development. Significantly, depending on multiple factors (e.g., cell age, history of previous cell sickling, cell hemoglobin concentration, cell size, etc.) cells containing hemoglobin S may have different propensity to sickle. Such variability would be further enhanced by treatments affecting internal red blood cell hemoglobin composition (e.g., introduction of hemoglobin F) or ability of hemoglobin molecules to bind to each other (polymerize) as is the case with e.g., Oxbryta treatment. Red blood cell transfusion therapy introduces to the patient previously collected and stored donor red blood cells containing hemoglobin A (normal hemoglobin, often abbreviated HbA or Hb-A) that does not sickle. Despite recent advances in drug development, this therapy remains one of most often used methods to managing sickle cell disease.

Red blood cell transfusion can be performed with pheresis. The public and individual cost of sickle cell disease is exceptionally high. The annual costs estimate for sickle cell disease treatment range from $50,000/child to $300,000/adult, with a life-time cost of care of $9M per individual. Sickle cell disease related annual cost of care in the United States exceeds $10B. Chronic red blood cell transfusion therapy, the present treatment of choice for more severe sickle cell disease patients, including about 10,000 children, has an estimated annual transfusion-associated hospital costs have been reported at $20,000-$50,000 per sickle cell disease patient, with pediatric being at the lower end of the range due to lower contribution of chelation therapy, which alone contribute ˜40% to total therapy costs. Pediatric red blood cell costs also can vary from $200 to over $1,500 for rare type units, with insurance reimbursement sometimes lower than actual hospital costs. Reported hospital transfusion-related costs also typically do not include those for treatment of post-transfusion complications ranging from minor fever/allergy to life threatening conditions such as transfusion related acute lung injury (TRALI). Accounting even for a part of these complications can double transfusion-related costs.

Red blood cell transfusion therapy is an important but less than ideal treatment option for sickle cell disease patients. It can be performed as a direct transfusion or in association with phlebotomy or pheresis with the goal of maintaining total hemoglobin and/or hemoglobin A above predefined levels. When transfusions are insufficiently or infrequently performed, i.e. “under-transfusion” complications can occur due to high hemoglobin S being present between transfusions which can lead to increased sickling. Increasing the frequency of the transfusions can also be problematic. “Over-transfusion” incurs extra costs and can expose the patient to volume and iron overload and toxic effects of cell free hemoglobin, as well as to additional risks associated with transfusions themselves. Chronic transfusions predispose individuals with sickle cell disease to iron overload, alloimmunization and autoimmunization. Iron overload, common for chronically transfused patients, and exacerbated in sickle cell disease by the shortened red blood cell lifespan, is often addressed through expensive chelation therapy. Alloimmunization results in life-threatening delayed hemolytic transfusion reactions and the autoimmune hemolytic anemia arising from hyper-hemolysis. Even with antigen match, 29% of transfused pediatric sickle cell disease patients can develop clinically significant alloantibodies, and 8% can develop autoantibodies. For adults, the respective values were 47% and 10%.

Transfusions for treating sickle cell disease can be administered as either a simple transfusion, which involves the addition of donor blood cells without the removal of any of the patient's blood, or as an exchange transfusion where a patient's blood is removed and exchanged with donor blood. Simple transfusions increase blood oxygen-carrying capacity but at a risk of elevating blood viscosity (hyper-viscosity) due to elevation of total hemoglobin levels. There is also an increased risk of transfusion associated volume overload (TACO) that can occur in patients receiving large volume of transfused blood especially over a short period of time, and pulmonary edema can develop as a result. The risks of TACO are elevated with underlying cardiovascular or renal disease. Because of these risks, simple transfusions are mostly performed in cases of acute, severe anemia that needs to be treated quickly. The risks of TACO and blood hyper-viscosity can be reduced by the use of exchange transfusion, which is the procedure most often used when the need for a transfusion is chronic, when a significant amount of blood needs to be transfused, and when the volume overload may lead to particularly elevated risks such as with some pediatric patients. The exchange transfusion has the additional benefit of increasing both blood oxygen-carrying capacity and reducing the fraction of hemoglobin S-containing cells in circulation since hemoglobin S-containing blood is removed from the patient.

With exchange transfusion, hemoglobin A levels can be safely maintained at a high level. Exchange transfusions can be performed as a manual procedure or as an automated procedure using an apheresis machine. Manual exchanges are performed using repeated alternating isovolumetric phlebotomy and blood transfusion. This can be a useful procedure, particularly in the acute situation to enable increase in hemoglobin A-containing cells and oxygen-carrying capacity with concurrent removal of hemoglobin S-containing red cells to prevent hyperviscosity. However, the process is time-consuming and needs skilled staff and constant medical supervision during the procedure. Automated red cell exchange also involves the replacement of red blood cells of a patient with sickle cell disease with donor red blood cells. The automated red cell exchange is performed by an apheresis machine and is a reasonably rapid procedure faster than the equivalent manual process, taking only 90 to 120 minutes, and can be performed in children as young as 5 years. Its other main advantage is the decreased rate of iron loading associated with this procedure, with a reduction of iron loading of ˜85% compared with simple transfusion. Automated red cell exchange is well tolerated in patients with sickle cell disease and results in good control of hemoglobin S without an increase in viscosity.

There are inherent disadvantages and problems with performing transfusions, no matter which type of transfusion is used. First is the need for and cost of securing suitable donor blood. Second, there is the risk to the patient due to transfusion-related complications, such as one or more of hemolytic transfusion reactions, non-immune hemolysis, various allergic and anaphylactic reaction, transfusion-related acute lung injury, febrile nonhemolytic transfusion reaction, post-tranfusion purpura, and infections like Hepatitis, West Nile Virus, and HIV. In addition, blood transfusions are typically associated with both immediate (up to 25 percent of transfused cells within the first 24 hours post transfusion as per FDA guidelines) and delayed (while the cells are in circulation) cell hemolysis. Such hemolysis depends on factors such as donor blood properties, manufacturing methods or blood donor processing methods, and stored blood properties over time.

There is therefore a need for an improved system and method for treating sickle cell disease. There is further a need for a system and method for treating a patient with sickle cell disease that reduces the need for donor blood. There is further a need for a system and method for treating a patient with sickle cell disease that offers benefits to the patient over receiving multiple blood transfusions.

### SUMMARY

The present invention satisfies these needs. In one aspect of the invention, an improved system and method for treating sickle cell disease is provided.

In another aspect of the invention, a system and method is provided for treating a patient with sickle cell disease in a manner that reduces the need for donor blood.

In another aspect of the invention, a system and method is provided for treating a patient with sickle cell disease in a manner that offers benefits to the patient over receiving multiple blood transfusions.

In another aspect of the invention, a system and method is provided for treating a patient with sickle cell disease by removing blood from the patient, treating the blood to decrease the fraction of red cells in the blood that are sickled, and reintroducing the treated blood back into the patient.

In another aspect of the invention, a system and method is provided for treating a patient with sickle cell disease by removing blood from the patient, treating the blood to decrease the fraction of red cells in the blood that are sickle-prone in that they are or may become sickled, and reintroducing the treated blood back into the patient.

In another aspect of the invention, a system and method is provided for treating a patient with sickle cell disease by removing blood from the patient, treating the blood to decrease the fraction of red cells in the blood that contain Hemoglobin S, and reintroducing the treated blood back into the patient.

In another aspect of the invention, a system and method is provided for treating a patient with sickle cell disease by removing blood from the patient, treating the blood to decrease the fraction of red cells in the blood that contain Hemoglobin S or any Hemoglobin variant that contains a single nucleotide replacement in the sixth amino acid of the Beta-Hemoglobin chain from glutamic acid to valine (Glu6Val).

In another aspect of the invention, a system and method is provided for treating a patient with sickle cell disease by removing blood from the patient, treating the blood to induce sickling of red blood cells in the blood that are sickle-prone, separating out at least a portion of the sickled red blood cells, and reintroducing the treated blood back into the patient with a reduced fraction of sickle-prone red blood cells.

In another aspect of the invention, a system and method is provided for treating a patient with sickle cell disease by removing blood from the patient, treating the blood to induce sickling of red blood cells in the blood that are sickle-prone by deoxygenating hemoglobin, separating out at least a portion of the sickled red blood cells, and reintroducing the treated blood back into the patient with a reduced fraction of sickle-prone red blood cells.

In another aspect of the invention, a system and method is provided for treating a patient with sickle cell disease by removing blood from the patient, treating the blood to induce sickling of red blood cells in the blood that are sickle-prone by enzymatic oxygen consumption, separating out at least a portion of the sickled red blood cells, and reintroducing the treated blood back into the patient with a reduced fraction of sickle-prone red blood cells.

In another aspect of the invention, a system and method is provided for treating a patient with sickle cell disease by removing blood from the patient, treating the blood to induce sickling of red blood cells in the blood that are sickle-prone by photochemical modification, separating out at least a portion of the sickled red blood cells, and reintroducing the treated blood back into the patient with a reduced fraction of sickle-prone red blood cells.

In another aspect of the invention, a system and method is provided for treating a patient with sickle cell disease by removing blood from the patient, treating the blood to induce sickling of red blood cells in the blood that are sickle-prone by gas exchange, separating out at least a portion of the sickled red blood cells, and reintroducing the treated blood back into the patient with a reduced fraction of sickle-prone red blood cells.

In another aspect of the invention, a system and method is provided for treating a patient with sickle cell disease by removing blood from the patient, treating the blood to induce sickling of red blood cells in the blood that are sickle-prone by chemically-induced oxygen scrubbing, separating out at least a portion of the sickled red blood cells, and reintroducing the treated blood back into the patient with a reduced fraction of sickle-prone red blood cells.

In another aspect of the invention, a system and method is provided for treating a patient with sickle cell disease by removing blood from the patient, treating the blood to induce sickling of red blood cells in the blood that are sickle-prone by using natural deoxygenation, separating out at least a portion of the sickled red blood cells, and reintroducing the treated blood back into the patient with a reduced fraction of sickle-prone red blood cells.

In another aspect of the invention, a system and method is provided for treating a patient with sickle cell disease by removing blood from the patient, treating the blood to decrease the fraction of red cells in the blood that are sickle-prone in that they are or may become sickled, separating out at least a portion of the sickle-prone red blood cells, and reintroducing the treated blood back into the patient.

In another aspect of the invention, a system and method is provided for treating a patient with sickle cell disease by removing blood from the patient, treating the blood to decrease the fraction of red cells in the blood that are sickle-prone in that they are or may become sickled, separating out at least a portion of the sickle-prone red blood cells by filtration, and reintroducing the treated blood back into the patient.

In another aspect of the invention, a system and method is provided for treating a patient with sickle cell disease by removing blood from the patient, treating the blood to decrease the fraction of red cells in the blood that are sickle-prone in that they are or may become sickled, separating out at least a portion of the sickle-prone red blood cells by density separation, and reintroducing the treated blood back into the patient.

In another aspect of the invention, a system and method is provided for treating a patient with sickle cell disease by removing blood from the patient, treating the blood to decrease the fraction of red cells in the blood that are sickle-prone in that they are or may become sickled, separating out at least a portion of the sickle-prone red blood cells by cell adhesion, and reintroducing the treated blood back into the patient.

In another aspect of the invention, a system and method is provided for treating a patient with sickle cell disease by removing blood from the patient, treating the blood to induce sickling of red blood cells in the blood that are sickle-prone, separating out at least a portion of the sickled red blood cells by filtration, and reintroducing the treated blood back into the patient with a reduced fraction of sickle-prone red blood cells.

In another aspect of the invention, a system and method is provided for treating a patient with sickle cell disease by removing blood from the patient, treating the blood to induce sickling of red blood cells in the blood that are sickle-prone, separating out at least a portion of the sickled red blood cells by density separation, and reintroducing the treated blood back into the patient with a reduced fraction of sickle-prone red blood cells.

In another aspect of the invention, a system and method is provided for treating a patient with sickle cell disease by removing blood from the patient, treating the blood to induce sickling of red blood cells in the blood that are sickle-prone, separating out at least a portion of the sickled red blood cells by cell adhesion, and reintroducing the treated blood back into the patient with a reduced fraction of sickle-prone red blood cells.

In another aspect of the invention, a system and method is provided for treating a patient with sickle cell disease by removing blood from the patient, treating the blood to decrease the fraction of red cells in the blood that are sickle-prone in that they are or may become sickled, storing the treated blood, and reintroducing the stored treated blood back into the patient.

In another aspect of the invention, a system and method is provided for treating a patient with sickle cell disease by removing blood from the patient, treating the blood to decrease the fraction of red cells in the blood that are sickle-prone in that they are or may become sickled, and reintroducing the treated blood back into the patient along with the introduction of stored blood.

In another aspect of the invention, a system and method is provided for treating a patient with sickle cell disease by removing blood from the patient, treating the blood to decrease the fraction of red cells in the blood that are sickle-prone in that they are or may become sickled, and reintroducing the treated blood back into the patient along with the introduction of stored blood, wherein the stored blood in introduced in an amount selected in relation to the treatment of the patient's blood.

In another aspect of the invention, a method for treating sickle cell disease comprises removing blood from a patient with sickle cell disease; treating the removed blood to decrease the fraction of sickle-prone cells in the removed blood; reintroducing the treated blood back into the patient, wherein the treated blood that is reintroduced back into the patient has a reduced number of sickle-prone red blood cells when compared to the blood removed from the patient.

In another aspect of the invention, a method for treating sickle cell disease comprises removing blood from a patient with sickle cell disease; inducing sickling of at least some sickle-prone cells in the removed blood; removing at least some sickled cells from the sickling-induced blood; and reintroducing the blood with the removed sickled cells back into the patient, wherein the reintroduced blood has a reduced number of sickle-prone red blood cells when compared to the blood removed from the patient.

In another aspect of the invention, a sickle cell disease treatment system comprises a line adapted to receive blood from a patient; a blood treatment unit in communication with the line adapted to receive blood from a patient, the blood treatment unit comprising: a sickling inducement unit adapted to receive blood from the patient and induce sickling of sickle-prone cells in the blood from the patient; and a sickled cell separation unit adapted to remove sickled cells from the blood received from the sickling inducement unit; and a line adapted to reintroduce treated blood from the blood treatment unit back into the patient, wherein the reintroduced blood has a reduced number of sickle-prone red blood cells when compared to the blood removed from the patient.

### BRIEF DESCRIPTION OF THE DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic flow chart of a method for treating sickle cell disease according to one version of the invention;

FIG. 2 is a schematic flow chart of a method for treating sickle cell disease according to another version of the invention;

FIG. 3 is a schematic flow chart of a method for treating sickle cell disease according to another version of the invention;

FIG. 4 is a schematic flow chart of a method for treating sickle cell disease according to another version of the invention;

FIG. 5A is a schematic flow chart of a method for treating sickle cell disease according to another version of the invention of FIG. 4;

FIG. 5B is a schematic flow chart of a method for treating sickle cell disease according to another version of the invention of FIG. 4;

FIG. 5C is a schematic flow chart of a method for treating sickle cell disease according to another version of the invention of FIG. 4;

FIG. 5D is a schematic flow chart of a method for treating sickle cell disease according to another version of the invention of FIG. 4;

FIG. 5E is a schematic flow chart of a method for treating sickle cell disease according to another version of the invention of FIG. 4;

FIG. 6A is a schematic flow chart of a method for treating sickle cell disease according to another version of the invention of FIG. 3;

FIG. 6B is a schematic flow chart of a method for treating sickle cell disease according to another version of the invention of FIG. 3;

FIG. 6C is a schematic flow chart of a method for treating sickle cell disease according to another version of the invention of FIG. 3;

FIG. 7A is a schematic flow chart of a method for treating sickle cell disease according to another version of the invention;

FIG. 7B is a schematic flow chart of a method for treating sickle cell disease according to another version of the invention;

FIG. 7C is a schematic flow chart of a method for treating sickle cell disease according to another version of the invention;

FIG. 8A is a schematic flow chart of a method for treating sickle cell disease according to another version of the invention;

FIG. 8B is a schematic flow chart of a method for treating sickle cell disease according to another version of the invention;

FIG. 9A is a schematic diagram of a system for treating sickle cell disease according to one version of the invention;

FIG. 9B is a schematic diagram of another version of a system for treating sickle cell disease;

FIG. 9C is a schematic diagram of another version of a system for treating sickle cell disease;

FIG. 9D is a schematic front view of a component of the system of FIG. 9A; and

FIG. 9E is a schematic side view of the component of FIG. 9D.

### DESCRIPTION

The present invention relates to a system and method for treating a patient with sickle cell disease. In particular, the invention relates to a system and method for treating a patient with sickle cell disease by treating the patient's blood outside the patient's body. Although the system and method are illustrated and described in the context of being useful for treating sickle cell disease, the present invention can be useful in other instances. Accordingly, the present invention is not intended to be limited to the examples and embodiments described herein.

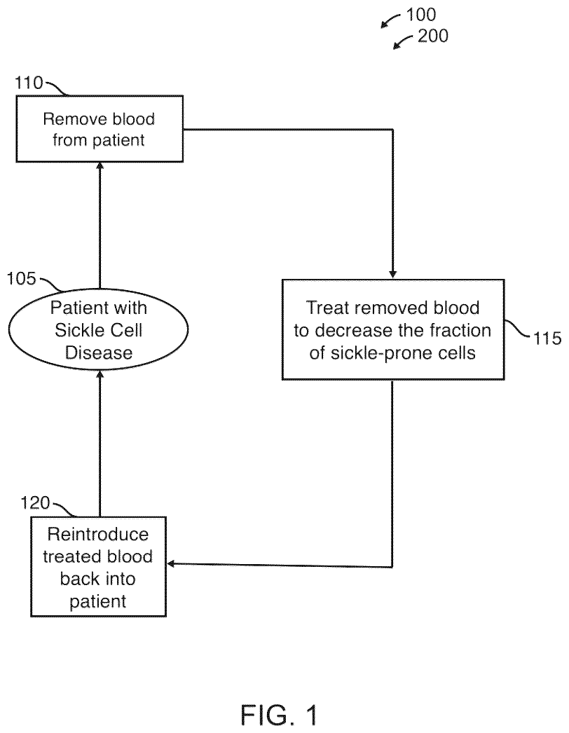

FIG. 1 shows a flow chart depicting a method for treating sickle cell disease 100 according to one version of the invention. In this method, a patient 105 with sickle cell disease has a portion of his or her blood removed 110 in conventional manner. The blood of the patient with sickle cell disease includes a mixture of many components, including various forms of red blood cells. The red blood cells can also be present in different forms, including both normal red blood cells, such as Hemoglobin A, and red blood cells that are or that are capable of becoming sickled. Thus, at any given time, the blood of the patient 105 with sickle cell disease can include red blood cells that are sickled, red blood cells that may become sickled, and red blood cells that do not have the likelihood of sickling. The invention as shown in FIG. 1 treats 115 the blood outside the body of the patient 105 to remove sickled red blood cells and/or red blood cells that may become sickled to lower the proportion or fraction of these deleterious red blood cells from the overall population of red blood cells in the patient's blood and to thereby increase the fraction of healthy red blood cells that are present in the patient's blood. The treated blood is then reintroduced 120 back into the patient so that the patient can enjoy the benefits of a higher fraction of red blood cells that are not sickled and/or that are less likely to sickle.

More specifically, the treatment 115 of the blood from the patient 105 is designed to decrease the fraction of sickle-prone cells in the blood. By sickle-prone cells it is meant red blood cells that are present in the blood of a patient with sickle cell disease that are either sickled or that have a propensity or an ability to become sickled under certain sickling conditions that might occur in a sickle cell disease patient. Sickle-prone red blood cells include Hemoglobin S, any Hemoglobin variant that contains a single nucleotide replacement in the sixth amino acid of the Beta-Hemoglobin chain from glutamic acid to valine (Glu6Val), and/or any other type of Hemoglobin or other composition that would give the red blood cell the ability to sickle under sickling conditions, including HbS-Beta thalassemia, HbS-Beta-Plus thalassemia, HBS-Beta-Zero thalassemia, HbSC, HbD-Punjab, HbO-Arab, and HbE, and the like. By sickle (and sickled, sickling, etc.) it is meant a change of shape or character of a red blood cell that results from the sickle cell disease state of the red blood cell, such as changes that occur due to polymerization of deoxygenated Hemoglobin S, or other Hemoglobin variant such as those mentioned above, inside the red blood cell. The change in shape often causes the red blood cell to take on an elongated, sickle, or crescent shape but shapes other than the norm disc shape of a non-sickled red blood cell are possible. Sickled red blood cells are more rigid, less bendable or pliable, and have decreased oxygen carrying ability when compared to normal red blood cells. It should be noted that sickle-prone red blood cells that are subjected to drug treatment might have a reduced propensity to sickle while under the treatment, but the propensity will increase over time as the drug effect lessens. As a result of the treatment step 115, the treated blood has fewer red blood cells that are prone to sickle than if the blood had not gone through the treatment step 115. The treated blood that is then reintroduced 120 back into the patient 105 so that the patient's blood has a decreased fraction of sickle-prone red blood cells. Accordingly, the patient 105 will experience improved health due to the removal of at least a portion of the sickle-prone red blood cells. The method of FIG. 1 can be performed once or multiple times in a loop. The method of FIG. 1 can be performed as needed or at regular periods of time, depending on the condition of the patient 105. By fraction of red blood cells it is meant a percentage or proportion of one or more types of red blood cells present of the entire volume of red blood cells in a particular amount of blood. For example, a fraction of sickle-prone red blood cells would be the percentage of total red blood cells that are either sickled or that may become sickled, as discussed above, whereas a fraction of sickled red blood cells would be the percentage of total red blood cells that are actually sickled at a particular time. The fractional composition of red blood cells present in a collection or sample of blood can be tested and determined by any conventional method, such as by using commercially available electrophoresis or HPLC (high performance liquid chromatography) systems. Sickling that occurs can also or alternatively be measured by methods such as visual observation through a microscope or a digital imaging device, by spectroscopic methods, such as light scattering or absorption, and the like.

The fewer red blood cells that are sickled within a patient's blood, the healthier the patient 105 will be and feel. Many of the deleterious effects of sickle cell disease are as a result of the actual sickling of the red blood cells. For example, with reduced sickling, a patient 105 with sickle cell disease is less likely to experience one or more of pain, vaso-occulsion, stroke, acute chest syndrome pulmonary hypertension, vaso-occlusive crisis, stroke, acute chest syndrome, pulmonary hypertension, splenic dysfunction, and renal dysfunction. In one version, the method of FIG. 1 removes a portion of the red blood cells that are actually sickled. This version provides an immediate effect on the health of the patient since there are fewer sickled red blood cells in the patient's blood. In another version, the method of FIG. 1 removes at least a portion of sickled red blood cells and at least a portion of red blood cells that are prone to sickle but are not yet sickled. This version provides both an immediate effect and an extended health effect for the patient since by removing red blood cells that may become sickled, the reduction in overall fraction of sickled red blood cells in the patient's blood can be prolonged. Without the removal of at least a portion of sickle-prone red blood cells that are not yet sickled, those sickle-prone red blood cells might sickle soon after the reintroduction of blood back into the patient 105. By performing the method 100 of FIG. 1 periodically, the patient's blood can be maintained and controlled at a composition with a reduced fraction of red blood cells that have a high propensity to sickle.

The invention of FIG. 1 is thus able to improve the condition of a sickle cell disease patient's blood by reducing the percentage of sickle-prone red cells in the blood, and the invention of FIG. 1 is able to accomplish this improvement with no or with at least a reduced use of transfused blood, as will be discussed. Reducing the need for transfused blood provides several advantages. For example, there is a reduced need for and cost of securing suitable donor blood. In addition, there is a reduction in the risk to the patient from transfusion-related complications, such as one or more of hemolytic transfusion reactions, non-immune hemolysis, various allergic and anaphylactic reaction, transfusion-related acute lung injury, febrile nonhemolytic transfusion reaction, post-tranfusion purpura, and infections like Hepatitis, West Nile Virus, and HIV. Furthermore, there is a reduction in the amount of hemoglobin released from lysed red blood cells into plasma due to hemolysis of transfused red blood cells.

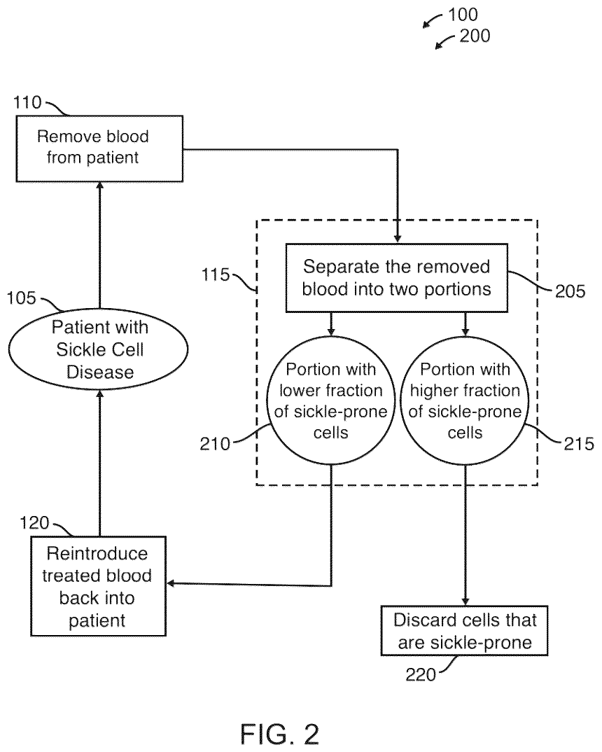

A particular version of the method for treating a patient with sickle cell disease 100 is shown in FIG. 2. The method of FIG. 2 is a sickle-prone cell separation method 200 that separates out or removes at least a portion of sickle-prone cells from the blood before the blood is reintroduced back to the patient 105. In this version, the step of treating 115 the blood to decrease the fraction of sickle-prone red blood cells involves the step of separating 205 the removed blood into two portions in order to be able to separate or segregate sickle-prone cells so they can be removed from the blood that is reintroduced 120 to the patient. This separation step 205 can be performed, for example, by filtering or otherwise separating the blood into a first portion 210 and a second portion 215. The separation step 205 results in a first portion 210 of treated blood that has a decreased fraction of sickle-prone red blood cells, and a second portion 215 of treated blood that has an increased fraction of sickle-prone red blood cells. In some cases, the second portion 215 may be mostly or substantially entirely sickle-prone red blood cells. The first portion 210 of the treated blood is reintroduced 120 back into the patient 105 so that the patient 105 has an overall reduced fraction of sickle-prone red blood cells. The second portion 215 can be discarded 220. By discarded it is meant that something is done with the separated second portion 215 and/or with the sickle-prone red blood cells other than reintroduction back into the patient. For example, the discarded red blood cells can be disposed of, analyzed, treated in some manner before reintroduction, or used in any other manner.

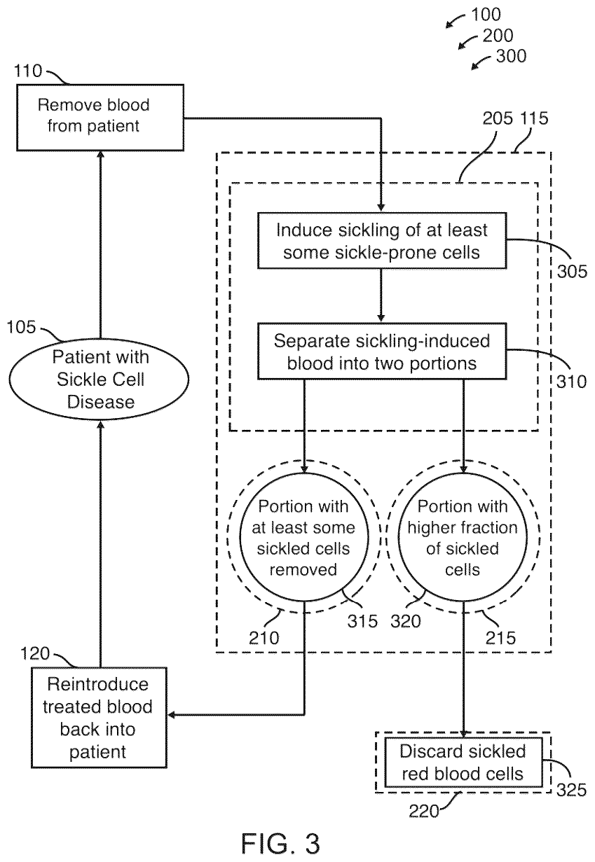

FIG. 3 shows a version of a method for treating a patient with sickle cell disease 100 in accordance with version of the invention of FIG. 2. In the version of FIG. 3, the sickle-prone cell separation method 200 is a sickle-prone cell separation by sickling inducement method 300. In this version, the step of treating 115 the blood to decrease the fraction of sickle-prone cells in the blood includes the step of inducing sickling 305 of sickle-prone red blood cells. By inducing sickling, the blood will include both red blood cells that were sickled prior to treatment and an additional set of red cells that are prone to sickle but were not sickled prior to the inducement step 305. The conditions of the inducement step 305 can be selected to cause a desired degree of sickling of the sickle-prone red blood cells. The inducement step 305 thus causes at least some red blood cells to sickle, and preferably a significant percentage, most, or nearly all. The treatment 115 then separates the sickling induced blood into two portions, as in FIG. 2. In the version of FIG. 3, the first portion with a lower fraction of sickle-prone cells 210 is made up of a portion 315 that has at least some of the sickled cells removed. The sickled cells that are removed can be cells that were already sickled and/or cells that sickled as a result of the inducement step 305. The second portion with a higher fraction of sickle-prone cells 215 in the version of FIG. 3 is a portion 320 that has a higher fraction of sickled cells. The portion 320 includes the sickled cells removed from the portion 315.

Thus, in the version of FIG. 3, the portion 210 of blood that now has a lower fraction of sickle-prone cells and that is reintroduced 120 to the patient achieves the lower fraction by the separation out of sickled cells, at least some of which were caused to sickle during the inducement step 305 of the treatment 115. The sickled red blood cells, whether they sickled prior to or during the inducement step 305, have a difference shape and/or deformability than the non-sickled red blood cells present in the blood, and this difference makes them separable. In one version, the step of separating 310 can comprise passing the blood containing both the sickled red blood cells and the non-sickled red blood cells through a filter. For example, the blood can be passed through a filter having pores from about 2 microns to about 10 microns, or from about 2 microns to about 6 microns, or about 5 microns, depending on the filter material and structure. The sickled red blood cells 315 will not pass as easily through the pores as the non-sickled red blood cells. Therefore, the blood that passes through the filter will have a reduced fraction of red blood cells that have sickled and thus a reduced fraction of sickle-prone red blood cells. This portion 315 can be reintroduced 120 back into the patient so that the patient will have blood with an overall reduction in the sickle-prone red blood cells, including red blood cells that were sickled when the blood was removed and/or red blood cells that were not yet sickled but would inevitably become so. The portion 320 with the sickled red blood cells can be discarded 325.

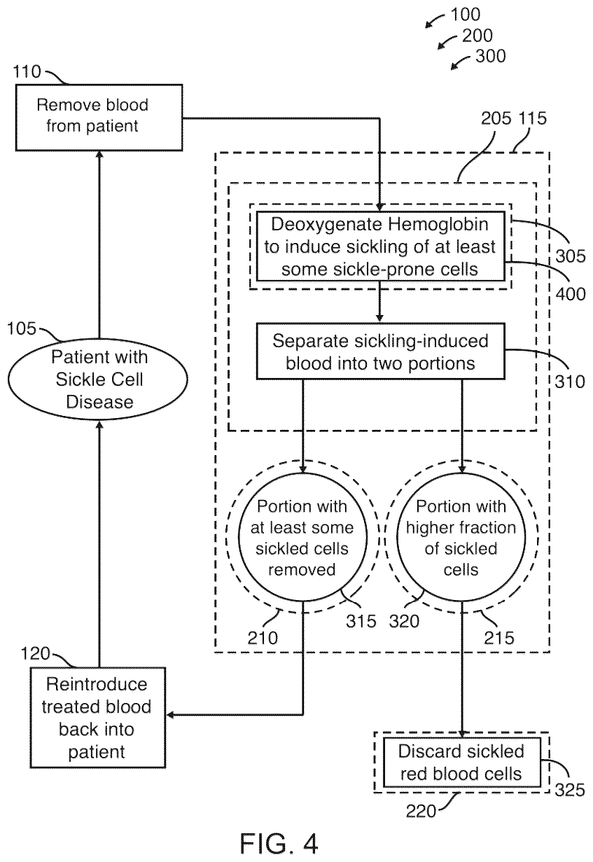

FIG. 4 shows a version of a method for treating a patient with sickle cell disease 100 in accordance with the method of FIG. 3. In the method of FIG. 4, the step of inducing sickling 305 of the sickle-prone red blood cells is performed by deoxygenating the hemoglobin 400 in the removed blood. Hemoglobin is a protein in the red blood cells that is responsible for carrying oxygen. When oxygen is removed from the hemoglobin of a red blood cell that is sickle-prone, the sickle-prone red blood cell has an increased tendency or likelihood to sickle. Therefore, by deoxygenating the hemoglobin in the red blood cells in the removed blood 105, the red blood cells more likely to sickle will sickle, and the red blood cells less likely to sickle will not sickle. The fraction of sickle-prone red blood cells that sickle during the deoxygenation step 400 can be controlled by the degree of deoxygenation applied. For example, a separation threshold can be established such that a desired fraction of red blood cells likely to sickle, would sickle and consequently would be separated out during the separation step 310. The threshold can be set so that a small proportion of the sickle-prone red cells are induced to sickle, or so that a large proportion of the sickle-prone red cells are induced to sickle, or anywhere in between.

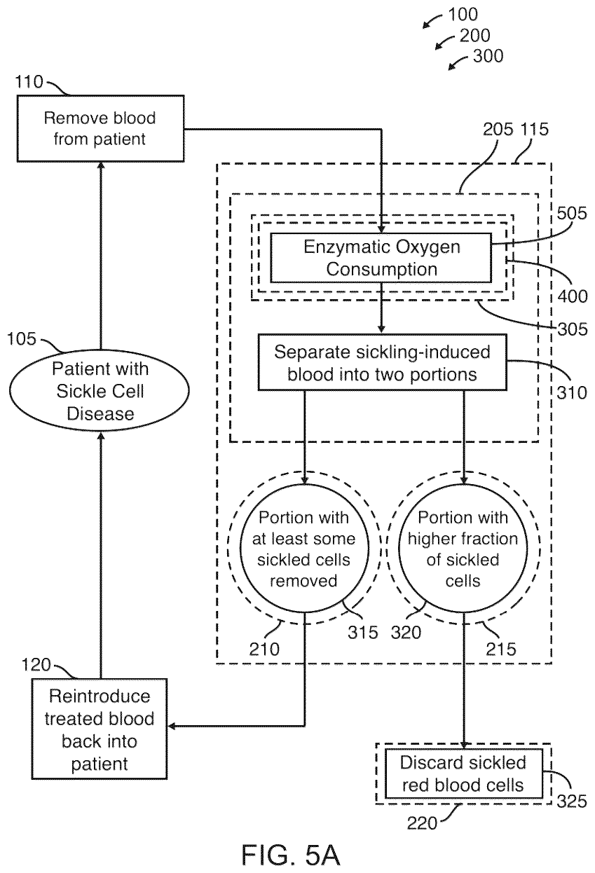

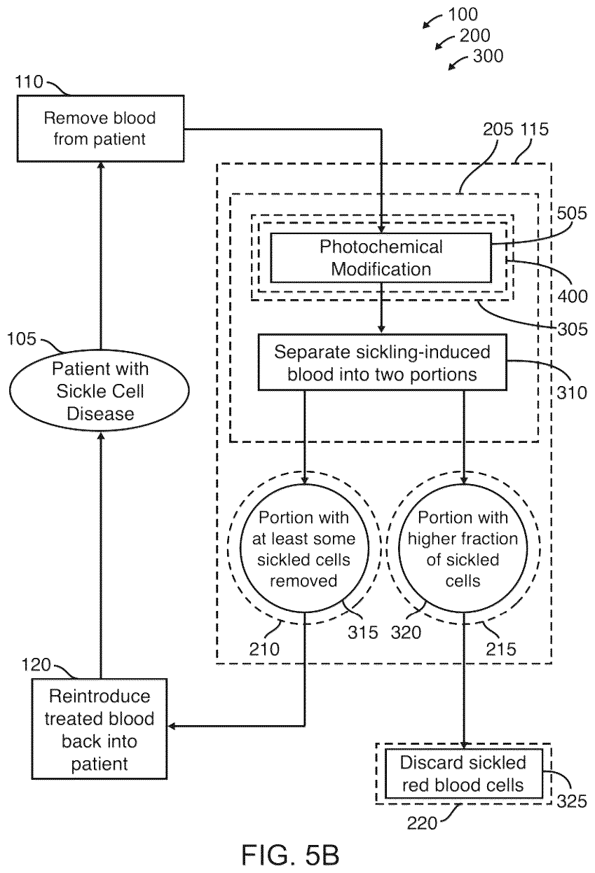

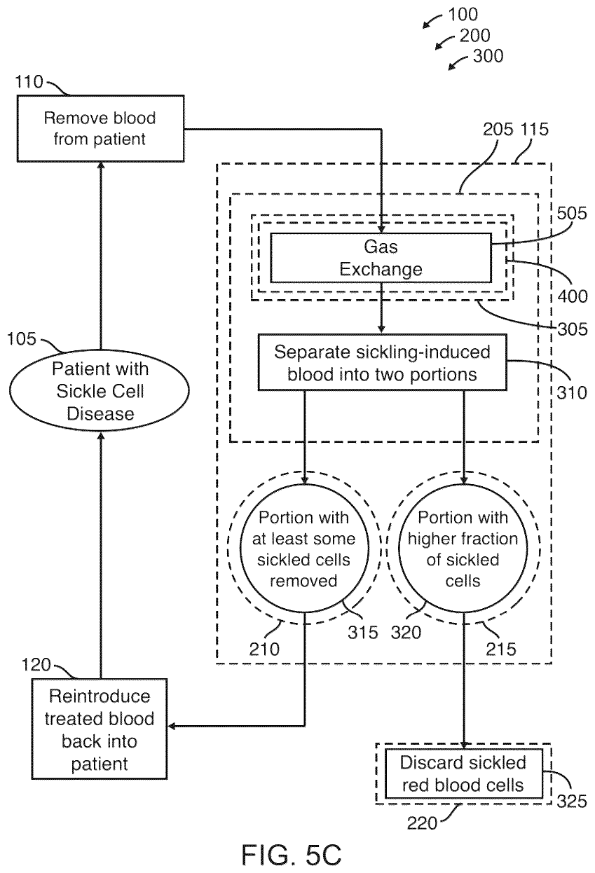

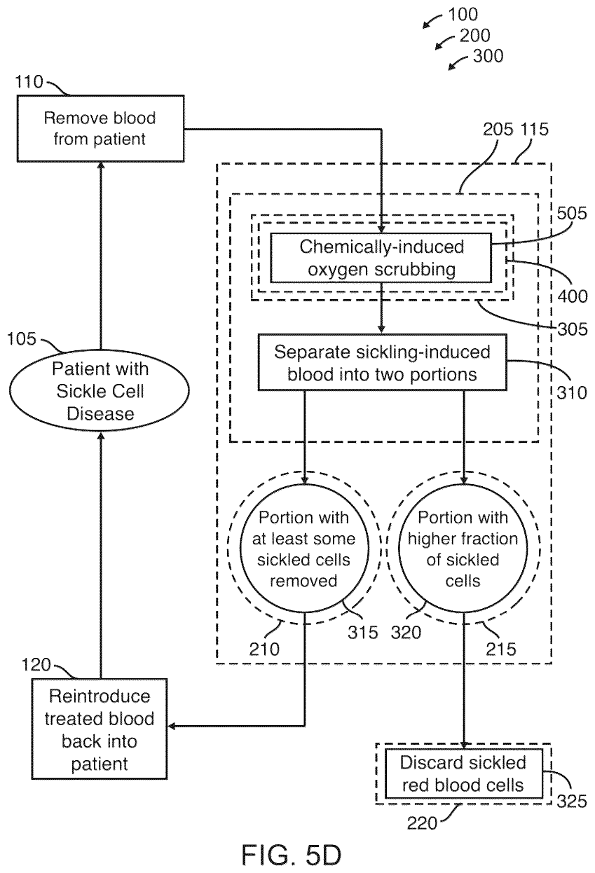

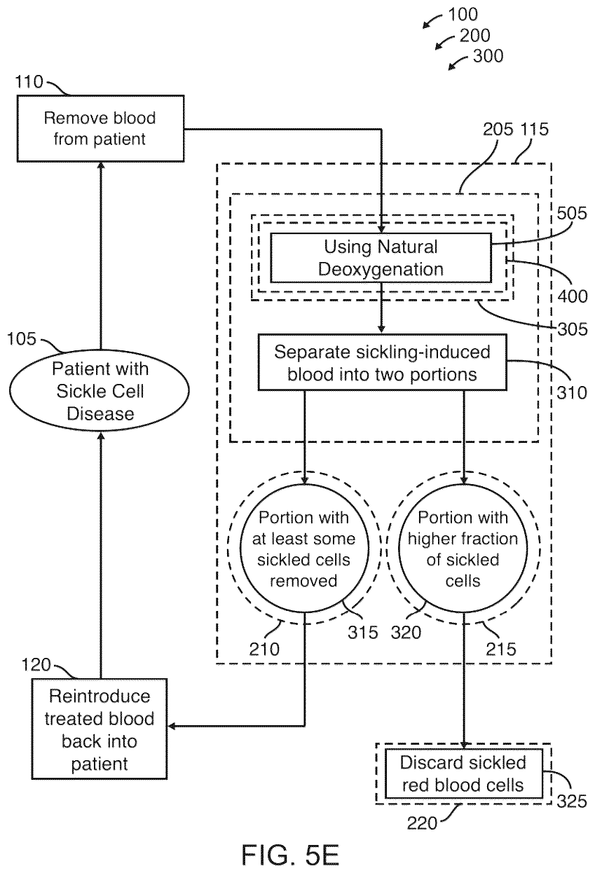

FIGS. 5A through 5E show various examples of the ways the deoxygenation step 400 can be performed. In one version of the invention, the step 400 of deoxygenating the hemoglobin in the method of FIG. 4 can be performed as shown in FIG. 5A. In this version, the step 400 of deoxygenation of the hemoglobin in the removed blood can be performed by a process of enzymatic oxygen consumption 505. By enzymatic oxygen consumption it is meant any step whereby an enzyme is brought into proximity, such as by being in the blood or in contact with the blood, with the removed blood with the enzyme being selected to initiate a chemical reaction between the enyme and oxygen thereby causing at least some of the oxygen that is associated with hemoglobin to become dissociated from the hemoglobin. This may also be described as enzyme-induced oxygen scrubbing. In accordance with this approach, the addition of an enzyme and an appropriate enzyme substrate to blood can initiate the enzymatic reaction that uses oxygen as a secondary substrate to generate product.

In one version, the enzyme for use in step 505 can comprise an oxidase. Oxidases are a prominent subclass of redox enzymes, which use oxygen either as oxidant or as electron acceptor. Multiple oxidase enzyme-substrate pairs can be used for removal of oxygen from the environment, as is known in the art. One such system is the combination of protocatechuate acid and protocatechuate dioxygenase (PCA/PCD). Other examples include glucose oxidase with glucose (Glu-Glu), galactose oxidase with galactose (Gal-Gal), and pyranose 2-oxidase with glucose (Pyr-Glu). Another example is a family of alcohol oxidases (AOX) that reduce primary and secondary alcohols to aldehydes and ketones, respectively. During this reaction, molecular oxygen is converted to hydrogen peroxide. Alcohol oxidases are not very specific in terms of substrates and can convert both primary and secondary alcohols to aldehydes and ketones, respectively. Such substrates include also methanol, ethanol, propanol, and butanol. Optionally, for many enzymatic oxygen scavenging systems, such as Glu-Glu, Gal-Gal, Pyr-Glu, or AOX systems, catalase can be added to the reaction medium for dismutation of hydrogen peroxide generated during the process the enzyme catalyzed oxygen removal. PCA/PCD system, does not generate reactive oxygen species, which makes it attractive for this step. The use of e.g., PCA/PCD or glucose oxidase (with catalase) may also be accompanied by pH stabilization as the reaction produces carboxylic acids. In another version, F420H2-oxidase is used in combination with reduced form of F420 (a deazaflavin derivative, which functions as electron carrier) to catalyze the four-electron reduction of O2 to 2 molecules of water. In this version. reactive oxygen species formation is prevented through a combination of difference reaction mechanisms blocking unwanted side-reaction between the catalytic intermediates and solvents.

In one version of the invention, the step 400 of deoxygenating the hemoglobin in the method of FIG. 4 can be performed as shown in FIG. 5B. In this version, the step 400 of deoxygenation of the hemoglobin in the removed blood can be performed by a process of photochemical modification 510 of hemoglobin. By photochemical modification of hemoglobin it is meant the application of light to oxygenated hemoglobin to cause at least a portion of the hemoglobin to become deoxygenated. An oxygenated form of hemoglobin (OxyHb) can be converted to deoxygenated form (deoxyHb) under both visible (VIS) and at a much higher quantum yield, under the ultraviolet (UV) light. The character of these photo-induced reactions typically remains the same regardless of the environment. That is, while the quantum yields may change, the reaction progression remains the same both in buffer solutions and in blood plasma. Moreover, it remains unchanged regardless of whether hemoglobin is free in solution or contained inside a red blood cell.

Whole blood, red blood cells separated from the blood, or red blood cells in buffer or storage solution, or packed RBC used for transfusion contain oxygen both dissolved in the medium and attached to hemoglobin molecules. Oxygen-free, closed-to-air, and open-to-air conditions can be differentiated. By open-to-air it is meant a condition when the medium (containing or not containing red blood cell and hemoglobin) is in contact with atmosphere and by extension in contact with atmospheric oxygen. This would allow for oxygen diffusion into the medium. Closed-to-air condition describes the conditions where blood is isolated from the atmosphere, but would contain the oxygen, bound and free in medium, it had before been placed in the close-to-air condition. In this condition there is no liquid-gas interface and no oxygen diffusion into the medium is possible. Note that in the case of open-to-air condition, unless there is agitation or mixing of the medium, oxygen will be very slow to diffuse from the liquid-gas interface into the medium (e.g., at room temperature the diffusion rate is about 1×10−5 cm/s, corresponding to about 0.3 mm propagation per minute). This implies that without mixing, if oxygen consumption occurs within the medium, volume of the medium sufficiently distanced from the liquid-gas interface can be considered to be closed-to-air if viewed on an appropriate time scale. Oxygen-free or anaerobic condition refers to a condition corresponding to anoxia or deep hypoxia when there is no or minimal amount of oxygen in the medium and by extension attached to hemoglobin. When open-to-air condition is present, especially with mixing enabling more efficient oxygen diffusion into medium, photochemistry of hemoglobin is significantly impacted by hemoglobin re-oxygenation by oxygen that is being diffused into the medium. Oxygen impact is significantly reduced when no diffusion is possible as in closed-to-air condition. In one version of the invention, we can assume whole blood or isolated red blood cells is in closed-to-air conditions. Irradiation of OxyHb free in solution or inside RBC, results in dose-and wavelength-dependent formation of alternative hemoglobin forms including oxidated Hb (metHb), deoxygenated Hb (DeoxyHb), and corboxy-Hb (COHb). The quantum yields of the reactions increase with the use of more high-energy (shorter wavelength) irradiation. Some of the quantum yields measured in human blood plasma are given in the table found in U.S. Provisional Patent Application 63/535,377 filed on Aug. 30, 2023 which is incorporated herein by reference.

Additionally or alternatively, illumination, and in particular shorter wavelength UV light (less than 320 nm) representing short UV-B and UV-C, can induce both red blood cell rupture (photo-induced hemolysis) and hemoglobin photo degradation. While hemolysis can be induced by UV-C irradiation (wavelength<300 nm), irradiation with light in UV-B, UV-A and VIS ranges (wavelength>300 nm) does not result in red blood cell lysis (at least up to the doses of 3×106 J/m2). In should be noted, that when UV-C irradiation is used, in case of photo-induced lysis of RBC, Hb released in buffer solution or blood plasma is predominantly in the COHb state.

Under irradiation in both VIS and UV ranges, hemoglobin photoconversion tend to follow the transition of oxyHb to MetHb and then to DeoxyHb, with the direct transition of OxyHb to DeoxyHb also being possible. Higher doses, especially at shorter wavelengths, leads to formation of COHb. Relative efficiency of Hb photo transformations in both buffer solutions and in plasma in close-to-air conditions is presented in the table found in U.S. Provisional Patent Application 63/535,377 filed on Aug. 30, 2023 which is incorporated herein by reference. Quantum yields of the reactions decline with the increase in the wavelength of irradiating light. In comparison with buffer solutions, in blood plasma the same irradiation dose leads to efficient formation of deoxyHb through metHb and directly to deox with an increase being about 3-5 times for UV-C irradiation and close to an order of magnitude increase for irradiation in the UV-A/VIS spectral range. Additionally, in plasma, higher dose of irradiation results in a formation of a significantly smaller follow-on COHb fraction. It can be seen that by appropriate selection of wavelength and dose of irradiation it is possible to transition a significant fraction of hemoglobin from oxyHb to deoxyHb form. While shorter wavelength may offer higher efficiency of the transformation, longer wavelengths allow for better defined approach even if higher total irradiation doses would be required.

In case of red blood cell containing hemoglobin S or any other hemoglobin variants capable of polymerization, Hb photo-induced deoxygenation would result in polymerization with the follow-up red blood cell sickling. Note, that in close-to-air environment, photo-generated hemoglobin can still be reoxygenated with the final amount of sickling dependent on relative magnitudes of polymerization and polymer melting delays, and on polymer formation and polymer melting rates. In red blood cells, such would be dependent on hemoglobin concentration and its composition with e.g., the presence of HbF introducing significant delay in polymerization and cell sickling further inhibited by the oxygen availability in the medium supporting hemoglobin reoxygenation.

It is also possible to select for or against generation of COHb. Notably, COHb had been proposed as a therapeutic agent for SCD. As CO binds to Hb much tighter than oxygen, presence of COHb fraction in red blood cells would reduce the amount of hemoglobin being deoxygenated and thus capable of polymerization. This is expected to have an inhibitory effect on red blood cell sickling, an effect potentially beneficial in prevention of vaso-occlusion. Note, that this would come at the expense of reduced blood oxygen carrying capacity and oxygen delivery to the tissues. In one version, systems optimized for photo-induced generation of carboxy form of hemoglobin can be used as a therapy by themselves, as well as in combination with cell separation and reinfusion. Short-wave irradiation (e.g., below 320 nm) allows for more efficient transition between hemoglobin forms, however it also induces hemolysis with hemoglobin being released into the medium. While cell-free hemoglobin would be detrimental for a patient, such undesirable effects can be avoided when post-irradiation, red blood cells are separated from plasma, buffer, or blood cell storage solution with such separated medium being replaced with e.g., buffer solutions supplemented with albumin. It should be noted, that hemolysis would be reduced when no oxygen is coming to the sample from the environment as in e.g., any closed to air circuit. This allows for a two-step process when initial irradiation is with a longer wavelength UV-A (e.g. 365 nm of the emission peak of a mercury lamp) or light in the visible range (e.g. 405-430 nm bands on a mercury lamp) to induce initial and partial hemoglobin deoxygenation with the follow up shorter UV irradiation (e.g., 250-320 nm range) to speed up the photoinduced Hb transformations with reduced amount of hemolysis. Irradiation with the longer wavelengths (in the near UV and visible spectrum (e.g. >400 nm, 400-550 nm) will induce progressively smaller (per unit of time) changes in hemoglobin form with increasing wavelength of irradiation. However, total irradiation time can be extended, and in a flow through system, this can be achieved by increasing the length of the path for the blood flow, thus extending the residence time of the blood or red blood cells under the irradiation.

In one version of the invention, the step 400 of deoxygenating the hemoglobin in the method of FIG. 4 can be performed as shown in FIG. 5C. In this version, the step 400 of deoxygenation of the hemoglobin in the removed blood can be performed by a process of gas exchange 515. The deoxygenation of hemoglobin in the removed blood, with resulting sickling of sickle-prone red blood cells, can be induced by gas exchange, for example, through a gas-permeable membrane, such as a membrane comprising polydimethylsiloxane (PDMS). Alternatively, the process can be performed through open-well gas exchange. As the oxygen diffusion rates in liquid water medium are very slow, without mixing of the medium, deoxygenation occurs equally slow. Use of PDMS membrane over a microfluidic channel, or any adjacent and other way arranged thin layer of medium, would allow for the rate of deoxygenation to be significantly increased. Deoygenation rates would also be increased if the deoxygenated medium is mixed, as would be the case for turbulent flows. Alternatively, gas exchange can be achieved through gas passing though the medium, as e.g. by “bubbling” the medium with nitrogen or argon. A bubbling process can sometimes damage blood cells and is thus more preferred and less damaging to soluble proteins and thus can be employed when after collection from a patient, red blood cells are separated and either patient plasma is being deoxygenated or if plasma, as during plasmapheresis, is being replaced with buffer most commonly supplemented with physiological concentration of albumin with or without additional supplements like calcium. Mixing red blood cells collected from the patient and separated from patient plasma (as e.g., in the phoresis procedure) with deoxygenated (e.g., by gas exchange) buffer solution would induce oxygen release from hemoglobin. Hemoglobin deoxygenation resulting from a single such procedure is unlikely to result is significant red blood cell sickling over what may already be present in patient blood. However, repeated cycles of red cell separation with the “reoxygenated” by oxygen released from hemoglobin medium being repeatedly replaced with fresh deoxygenated medium can be used to generate condition promoting red blood cell sickling with the amount of sickling potentially dependent of the number of medium exchange cycles. Alternatively, hemoglobin in red blood cells may be significantly deoxygenated in blood or separated red blood cells remain under the conditions promoting oxygen diffusion from the medium sufficiently long to account for oxygen release from hemoglobin. The speed of such process would be decreasing with increased hemoglobin's deoxygenation and with full hemoglobin deoxygenation likely being impractical due to time constraints. However, partial hemoglobin deoxygenation can still be induced by such an approach allowing to induce sickling in hemoglobin S containing red blood cells with higher propensity to sickle. Such cells can then be separated as detailed above with un-sickled red blood cells reintroduced to the patient and sickled cell discarded.

While microfluidics allows for faster oxygen exchange than open-well systems, the rates remain limited by diffusion through artificial membranes. Alternatively, diffusion rates can be increased by mixing the blood with an e.g., perfluorinated carrier oil as oxygen sink which allows for faster (0.1 to 0.5 sec) pO2 equilibration. However, achieving low oxygen concentration in blood by this method is at least problematic, if not completely impossible. There are also other gas-exchange techniques that also aim to reduce diffusion time, but they need for gas tanks, such as oxygen, nitrogen and/or argon tanks, and pressure regulators, often require control of sample dehydration, as well as retain the inherent complexity of changing deoxygenation levels and/or of controlling oxygen gradients in the target medium. Gas-exchange systems are also commercially available, albeit commonly used for blood oxygenation, not deoxygenation. Such systems implement multiple strategies for fastest and most efficient oxygenation, through gas exchange, on flow-through patient blood when such oxygenation support is required. Blood oxygenators are commonly used as part of the ECMO (Extracorporeal Membrane Oxygenation) circuits. When part of ECMO system, such units are connected to oxygen gad tanks, however if connected to e.g., nitrogen gas supply, they would allow for blood deoxygenation.

In one version of the invention, the step 400 of deoxygenating the hemoglobin in the method of FIG. 4 can be performed as shown in FIG. 5D. In this version, the step 400 of deoxygenation of the hemoglobin in the removed blood can be performed by a process of chemically-induced oxygen scrubbing 520. With this approach, one or more chemical reducing agents, such as sodium sulphate or metabisulphite is used to induce deoxygenation. An extensive number of such approaches for liquid sample deoxygenation in other fields have been used due to the advantages of simplicity of implementation, low cost and possibility for faster action. A possible disadvantage of the method is the generation of Reaction Oxygen Scpecies (ROS) as part of the oxygen scubbing chemical reaction able to induce cell damage and protein degradation. The detrimental impact of ROS generated during the reaction can be ameliated through the use of appropriate antioxydants (e.g., superoxide dismutase for superoxide or catalase for hydrogen peroxide). A derivation of the method uses reducing agent separated by a PDMS membrane eliminating direct sample contact with the agent with the inclusion of a diffusion step as in gas exchange methods.

In one version of the invention, the step 400 of deoxygenating the hemoglobin in the method of FIG. 4 can be performed as shown in FIG. 5E. In this version, the step 400 of deoxygenation of the hemoglobin in the removed blood can be performed by a process of using natural deoxygenation 525 that occurs in the blood that has been removed. For example, the removed blood can be removed from a vein of the patient 105 having sickle cell disease. The vein carries blood that has been deoxygenated by delivery of oxygen to the cells of the patient 105 in the patient's vascular system. In one version of this method, steps can be taken to reduce the removed blood's exposure to oxygen and/or to add deoxygenated anticoagulants or additive if long term storage is needed. Once reexposed to oxygen, at least a portion of the sickled cells will unsickle. Thus, in this version, a greater number of red blood cells that are likely to sickle can be separated than if a portion are allowed to unsickle. Alternatively, collected blood or isolated patient red blood cells can be placed in storage in closed-to-air conditions. This results in progressive deoxygenation, which in turn would lead to progressively increased sickling of red blood cells, which then can be separated. Increased duration under such closed-to-air condition would induce more severe deoxygenation leading to sickling of red blood cells with progressively lower propensity to sickle. The rate of such “natural” deoxygenation could significantly vary between the patients potentially necessitating monitoring of the process (e.g., through direct microscopic observation of cell sickling).

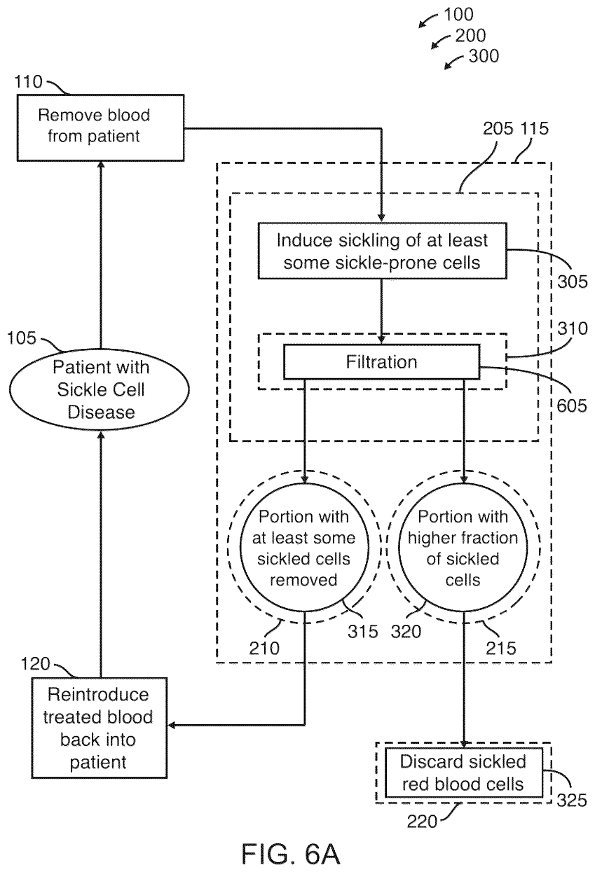

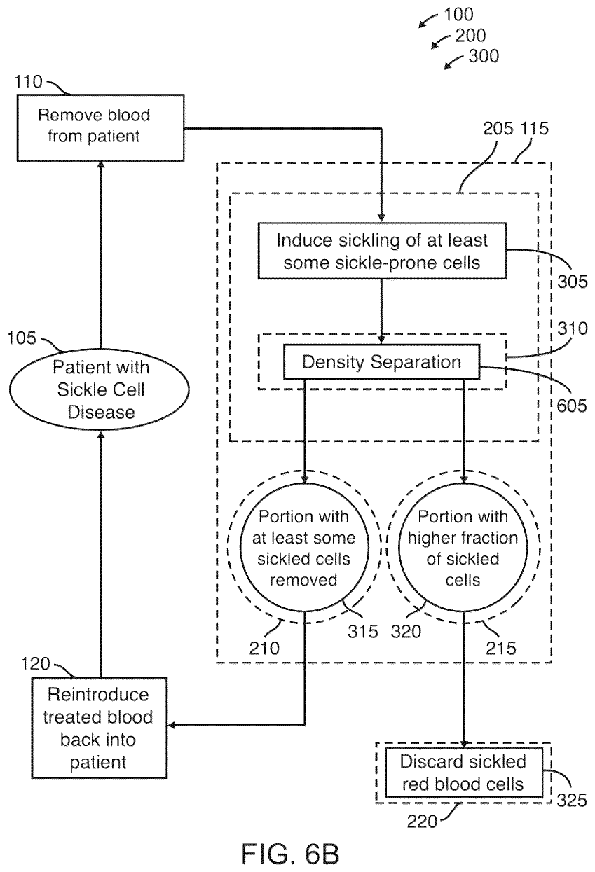

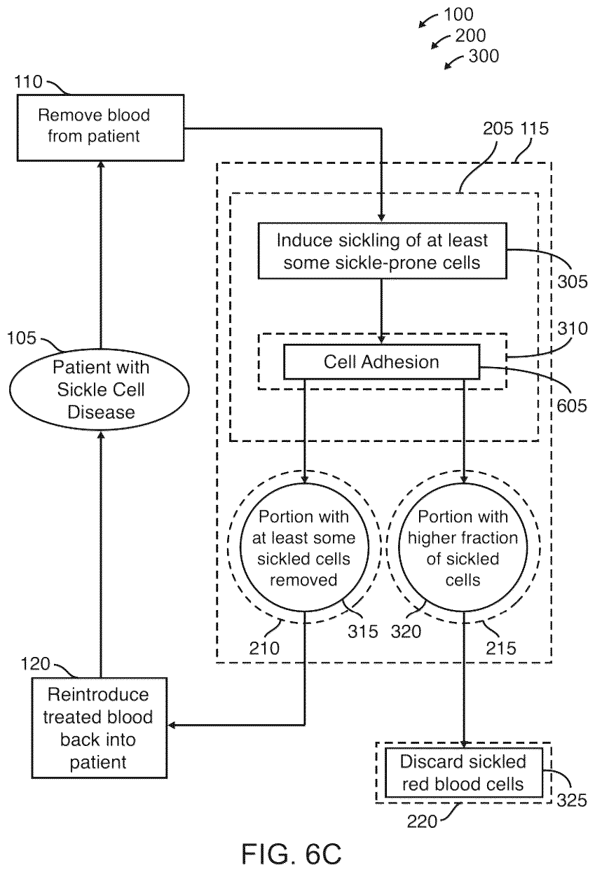

FIG. 6A shows a version of the invention of FIG. 3 and/or any of FIGS. 4 and 5A through 5E, where the step 310 of separating at least a portion of the sickled red blood cells from the rest of the blood includes the step of filtering 605 the sickled red blood cells from the rest of the blood. Morphological changes associated with sickling will result in sickled red blood cells with decreased deformability and altered shape, which will reduce sickled red blood cells' ability to traverse microcapillaries. These sickled red blood cells can thus be separated from non-sickled cells by filtering using, for example, a standard leukoreduction filter. The efficiency of filtering can be adjusted by changing the filter pore diameter and/or material. The size of the filter should be sufficient to allow efficient flow of the volume of the collected blood without significant flow rate reduction due to filter clogging by sickled red blood cells that are filtered out. Alternatively, a portion of filtered out sickled cells can be removed through backflow through the filter using patient's plasma or buffer solution (e.g., one of the storage solutions like AS1, AS3, or CPDA). The cells that had been filtered out are then discarded 320, and the cells that pass through the filter are reintroduced 120 back into the patient 105.

In one version, the filtration or sieving step 605 of FIG. 6A can be achieved using one or more filters or a single filter with one or more layers. Non-sickled red blood cells (discocytes) have a bi-conclave disk shape of approximately 7.5 to 8.7 μm in diameter and 1.7 to 2.2 μm in thickness. The cells are highly deformable and can pass through capillaries that are only 3-4 μm in diameter. Less deformable cells or cells with altered morphology (non-discoid; e.g., sickled cells of crescent or holly leaf morphological forms, spherocytes, etc.) would not be able to pass though smaller capillaries resulting in cell entrapment and consequent hemolysis. Such entrapment of sickled cells in the patient's body is a major contributing factor of sickle cell pain crisis and other complications associated with vaso-occlusion. Thus, in one version of the invention of FIG. 6A, the one or more filters can have a pore size of from about from about 2 microns to about 10 microns, or from about 2 microns to about 6 microns, or about 5 microns, depending on the filter material and structure. In another version, the one or more filters comprises multiple filters each having different pore sizes and/or the one or more layers comprises multiple layers each with different pore sizes. In this version, the filter comprises a filtering assembly with filters of progressively smaller pose sizes (e.g., with pore size progressively decreasing from 30 to 3 micron) can be used. The advantage of a single filter system is in its simplicity and the ability of potentially using existing commercial filtration systems even if not designed for similar application (e.g., commercial 4 μm LR filter would allow platelet and deformable red blood cells passage but retains leukocytes). Note, that leukoreduction is achieved through both the mechanical entrapment (sieving), which would be dependent on the size of the pores and of deformability of cells, and physical-chemical entrapment or adhesion of leukocytes to the material of the filter. Filters may have several layers with different diameter pores, which would them permit depth filtration with the larger pores being up to 30 μm. The filter pore size then determines the sieving of cells particles bigger than 30 μm retaining progressively smaller and less deformable cells at each layer. LR filters may also use materials designed to enhance leukocyte adhesion of use additional coatings for the same purpose. Such are not required in the application of the invention. LR filters are also designed for minimal retention of normal deformable discoid red blood cells, which can be an advantage.

FIG. 6B shows a version of the invention of FIG. 3 and/or any of FIGS. 4 and 5A through 5E, where the step 320 of separating at least a portion of the sickled red blood cells from the rest of the blood includes the step of separating the sickled red blood cells by density separation 610 from the rest of the blood. The density separation can be achieved, for example, by centrifugation using e.g. discontinuous or continuous Percoll gradients, by self-assembling step-gradients in density created by aqueous multiphase systems, by liquid-liquid separation, or the like.

FIG. 6C shows a version of the invention of FIG. 3 and/or any of FIGS. 4 and 5A through 5E, where the step 320 of separating at least a portion of the sickled red blood cells from the rest of the blood includes the step of separating the sickled red blood cells by a cell adhesion process 615 whereby sickled red blood cells will adhere to one or more selected substrates that interact with the red blood cells, such as biological endothelial substrates, such as p-Selectin, laminin, VAM1, and the like. Red blood cell adhesion to such substrates can be either static, meaning essentially irreversible cell adhesion to the substrate, or dynamic, meaning cells adhering to the substrate will after a time interval release from the substrate. Both static and dynamic adhesion would be dependent on various factors, such as medium composition (for example other molecules present in the environment of red cells that may support or induce adhesion or alternatively inhibit adhesion) and flow rate. These factors can each or together affect red blood cell adhesion. While the fraction of normal red blood cells is typically low, the adhesion conditions can be adjusted to enable a higher degree of adhesion for Hemoglobin S containing red blood cells.

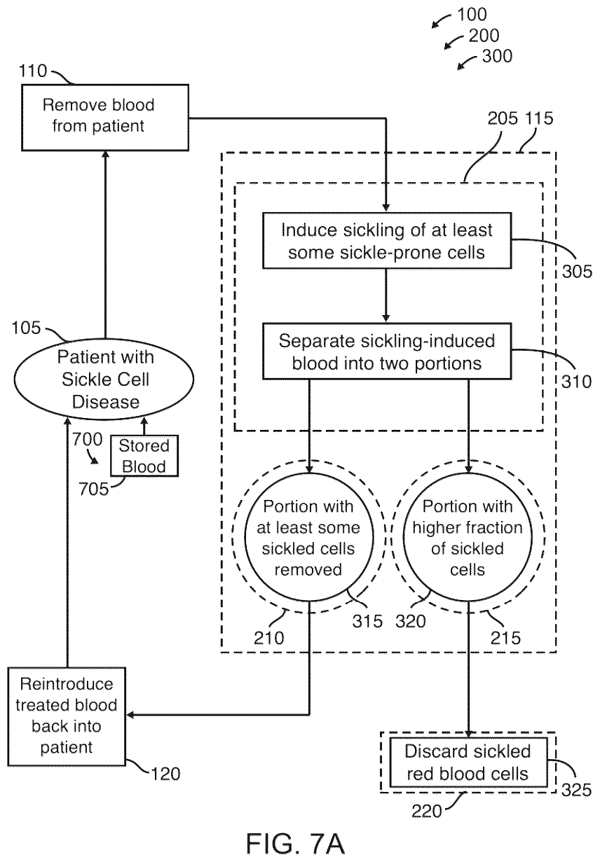

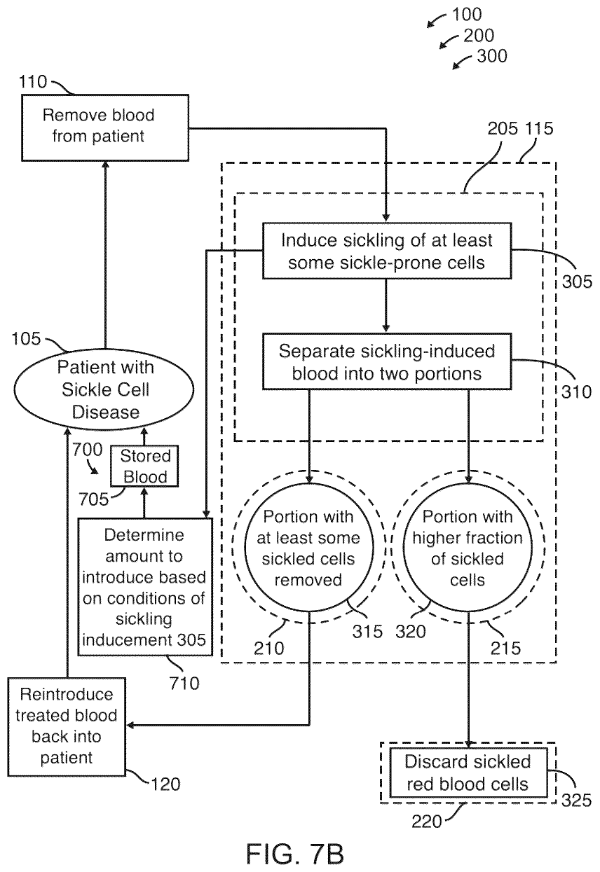

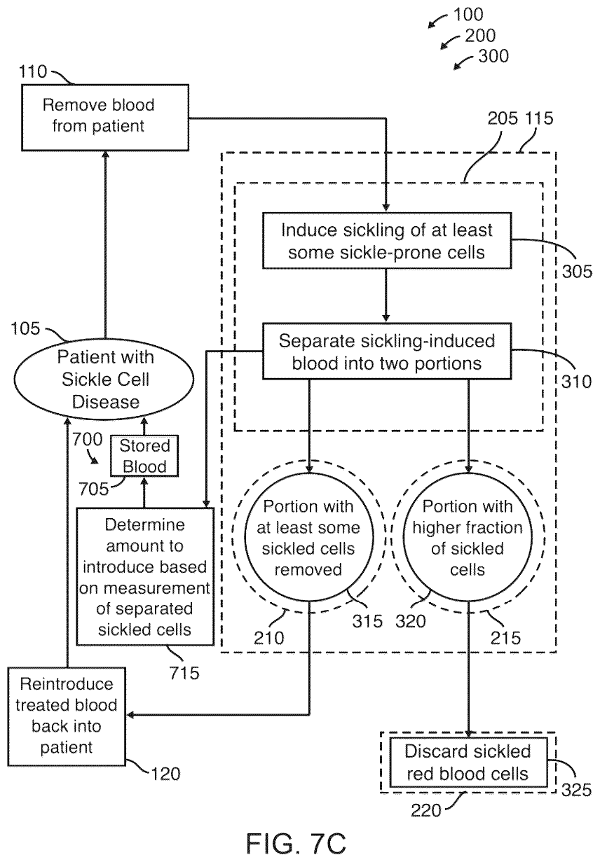

FIG. 7A shows a version of a method of treating a patient with sickle cell disease 100 in accordance with the method of FIG. 3, or any of the other disclosed versions, in which the process includes a blood transfusion process 700. In this version, the processes 100, 200, and/or 300 can be used in conjunction with the introduction of stored blood 705 into the patient 105. The stored blood 705 can be donor blood or other blood that is substantially absent red blood cells that contain hemoglobin S or are otherwise sickle-prone and can, in one version, be previously treated and stored blood from the patient 105. The process of FIG. 7A can be a conventional transfusion method and system that is supplemented by the treated blood 115.

In one version, such as shown in 7B and 7C, the introduction of stored blood 705 can be done to account for the volume of blood that is separated 310 and discarded 325 so that the overall blood volume does not decrease or decreases less than it would without the introduction of stored blood 705. In FIG. 7B, the amount of stored blood 705 to be introduced into the patient 105 is determined 710 or estimated by consideration of the conditions of sickling inducement in step 305. As discussed above, the conditions can be set to cause a small amount of sickling, a large amount of sickling, or anything in between. By knowing these conditions, a determination or estimate can be made as to how much sickling will occur and thus how much blood will be separated from the removed blood. The volume of stored blood to be introduced 705 can be selected in accordance with that determination or estimate. In the version of FIG. 7C, the volume of blood or the amount of red blood cells that is separated out of the removed blood is measured and the volume of stored blood (packed red blood cells) that is to be introduced into the patient 105 is determined 715 based on the measurement. In another version, the volume of stored blood 705 to be introduced can be a combination of the a consideration of the conditions of sickling inducement and a measurement of the separated and discarded blood.

In the version of FIGS. 7B and 7C, the volume of stored blood 705 to be introduced into the patient can be in relation to the volume removed by the treatment process 115. In one version, these volumes can be equally or substantially equal. Replacing the lost volume with an equal volume is particular useful with severely anemic patients. For patients on chronic transfusion or blood exchange who are not anemic, the volume may not be equal, and the goal may be to keep red blood cell and/or hemoglobin numbers approximately the same, particularly when the stored blood 705 is in concentrated form. Thus, the volume of stored blood 705 to be introduced in relation to the removed blood or components can vary. For example, the volume of stored blood 705 to be reintroduced can be (i) substantially equal to the lost volume, (ii) in an amount that substantially replaces the lost red blood cells, (iii) in an amount that substantially matched both the lost volume and the lost red blood cells, with stored plasma being used as needed, (iv) in an amount where the hemoglobin introduced is greater than the amount lost, or (v) any other amount selected in relation to the treatment process 115.

It should be noted that the word “blood” is used herein with its commonly usage in the art, such as when used with a blood transfusion, even if that which is being introduced is not actually blood. Only a portion of all transfusions are performed with actual blood, typically referred to as whole blood. Many transfusions are performed with red cell concentrates (processed donor red cells in storage solution). Unlike whole blood, such concentrates do not contain blood plasma or other cells (like white cells or platelets). The concentrates also have a much higher number of cells per ml than whole blood and are often referred to as packed red blood cells. In the version of FIGS. 7A, 7B, and 7C, the stored blood 705 that is introduced into the patient can take on any other these, or other, forms.

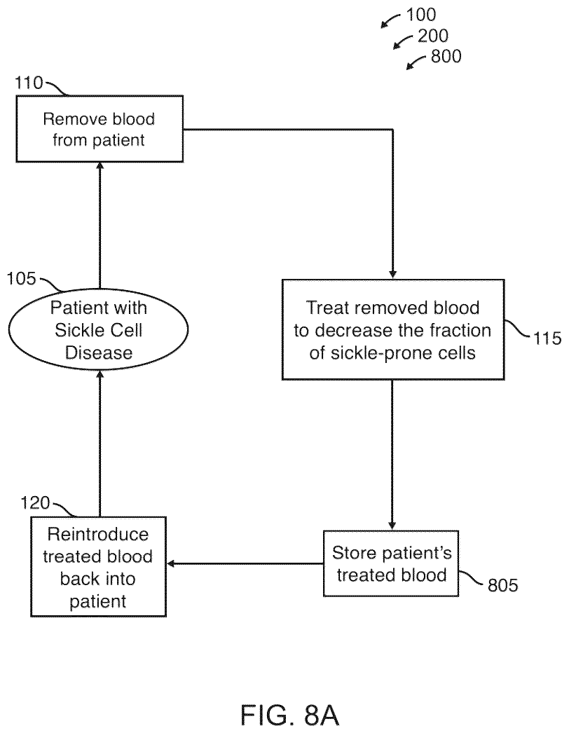

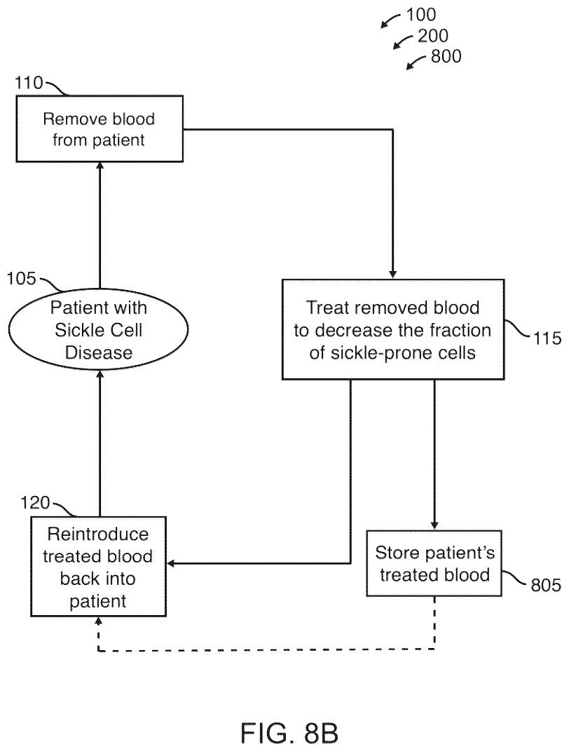

FIG. 8A shows a version of a method of treating a patient with sickle cell disease 100 in accordance with the method of FIG. 1, or any of the other disclosed versions, in which the process includes a treated blood storage process 800. In this version, the processes 100, 200, and/or 300 can be used to treat removed blood from the patient 105 and is then stored 805 before being reintroduced back into the patient 105, or into another patient, rather than being reinfused immediately back into the patient as in a blood exchange procedure. The storage can be in regular blood storage solution so that is can be reinfused at some later date, such as when needed to treat the patient's sickle cell disease or when blood may be otherwise needed by the patient. One treatment option would be reinfusion at the patient's following blood treatment procedure creating a chain process where at each procedure, which can be for example every three or four weeks, the patient's stored and treated blood is reinfused while new patient blood is being collected and treated and again stored. In another example, the stored and treated blood can be collected in anticipation of a patient's upcoming surgery or in case of emergency. FIG. 8B shows a version similar to the version of FIG. 8B but where a portion of the treated blood is immediately reintroduced 120 back into the patient 105 and a portion is pulled out and stored 805 for later reintroduction back into the patient 105, or other patient.

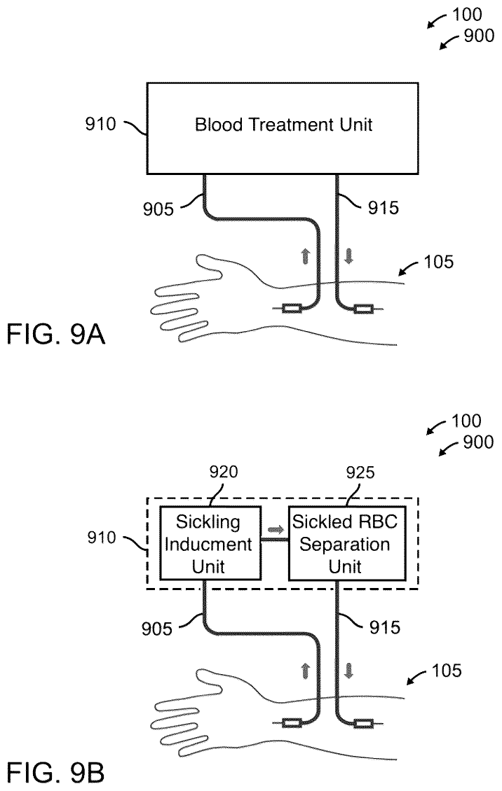

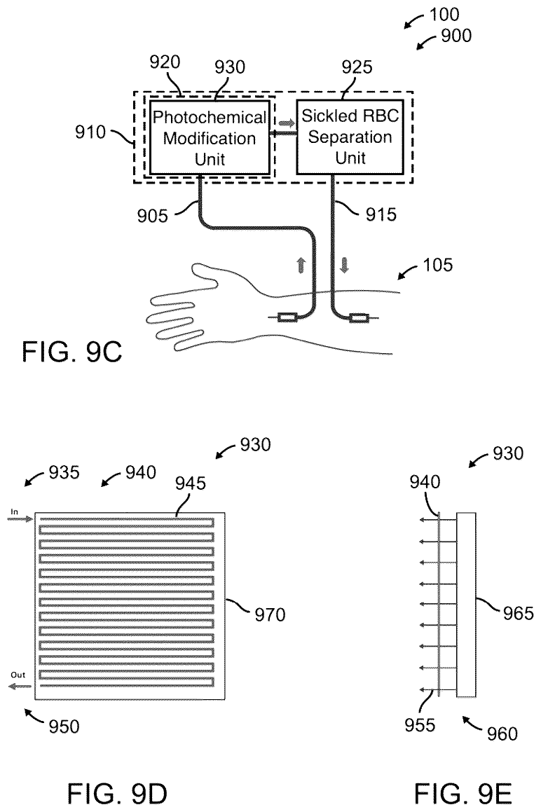

FIG. 9A shows a system 900 for treating a patient with sickle cell disease. The system 900 of FIG. 9A includes a line 905 adapted to receive blood from a patient 105 and deliver removed blood from the patient 105 to a blood treatment unit 910 that receives removed blood in accordance with process step 110 and treats the removed blood in accordance with any of the herein described treatment processes 115. Following treatment 115, the treated blood can be reintroduced 120 back into the patient via a line 915 adapted to reintroduce treated blood back into the patient 105. In the version shown in FIG. 9B, the blood treatment unit 910 is particularly designed to carry out the process of FIG. 3. Accordingly, the blood treatment unit 910 includes a sickling inducement unit 920 that can be used to cause at least a portion of the hemoglobin S containing red blood cells and/or the red blood cells with a propensity to sickle to sickle. For example, the sickling inducement unit 920 can include a hemoglobin deoxygenator, such as one or more of an enzymatic oxygen consumption unit, a photochemical modification unit, a gas exchange unit, a chemically-induced oxygen scrubbing unit, and the like. As also shown in FIG. 9B, the blood treatment unit 910 can also include a sickled red blood cell separation unit 925, such as one or more of a filtration unit, a density separation unit, a cell adhesion unit, and the like.

A particular version of a system 900 for treating a patient with sickle cell disease is shown in FIG. 9C. In this version, the blood treatment unit 910 includes a photochemical modification unit 930 as the sickling inducement unit 920. An example of the operation of the photochemical modification unit 930 is shown in FIGS. 9D and 9E. The blood removed from the patient 105 by, for example, phoresies or phlebotomy and that is to be treated enters the photochemical modification unit 930 at inlet 935. The blood that enters through the inlet 935 travels along a photochemical modification path 940 made up of channels 945 that carry the blood and then exits the unit through the outlet 950. The blood flowing through the path 940 can be whole blood, isolated red blood cells, and/or red blood cells in buffer or storage solution. Such path 940 can be implemented though enclosing the channels 945 in the path 940 in material transparent or with minimal absorbance to irradiation of the selected wavelength. For example, if UV irradiation in 350-370 nm range is selected, UV-T plastics could be used, and if e.g., 300-310 nm irradiation is selected UV-TC plastics could be used. Fused silica or quartz plates with low absorbance in both VIS and UV ranges can be used. Alternatively, the path 940 may be implemented using a UV-T/UV-TC tubing e.g., coiled in a spiral (not shown). The length of the path 940 and the flow rate would define the residence time of the cells in the path 940 and thus the time they are exposed to irradiation 955 from an irradiation source 960, such as light (UV, VIS, or combined) source 965. High intensity light source can be e.g., a short arc mercury lamp. Combined with the intensity of the light irradiation source 965, exposure time, would determine the cumulative dose of light irradiation received by the red blood cells. That, together with the wavelength or wavelengths of irradiation would determine the type and the progression of photo-induced changes of hemoglobin.

While the material between the sample in channels 945 and irradiation source 960 should be transparent to the wavelength or the range of wavelengths of radiation used for hemoglobin photo conversion, it can be simultaneously used to filter out the radiation with “undesirable” wavelengths. Considering the differences in quantum yields of the photo-induced reactions (Table 1 above), it is more important to filter out shorter wavelength radiation before it reaches the blood or red blood cells in solution. For example, if a mercury-based light source is used, and the band with maximum at 365 nm is used for photoconversion, bands with the longer wavelengths (>400 nm) would have relatively small effect on the process, however the bands with shorter wavelengths (e.g. 300-310 nm, or 254 nm) would, if not filtered out, drive the reactions instead of the 365 nm selected. Optimally, both shorter and longer wavelengths (relative to the spectrum range used) would be filtered out. Such filtering can be achieved using common band-bass filters or could be implementing by proper selection of the material for the enclosure 970 of channels 945 and path 940. Absorbance of high intensity irradiation, particularity of UV light, results in heating of absorbing material, necessitating the use of a cooling system. Such can be implemented e.g., through immersion of the blood (red blood cell) flow path or the whole enclosure 970 in a circulating low temperature water bath, by using cold air flow, or by any other means conventionally employed for temperature stabilization.

Regarding deoxygenation and associated cell sickling in all of the versions described above, hemoglobin polymerization is a process that depends on both the rate of deoxygenation and hemoglobin (mainly related hemoglobin S fraction) concentration within the red blood cell. If hemoglobin S fraction within the cell is low as it may be as a result of e.g., hydroxyurea or gene editing therapy, hemoglobin polymerization may still be possible, but would be, sometimes very significantly, delayed. Thus, the time the cells spent under deoxygenated conditions can be an important factor in the size of the fraction of cells that would sickle and later removed by the filtration system. Such time can be regulated e.g., by controlling the flow rate of blood or isolated red blood cell through the flow path 910, by introducing a delay between deoxygenation and sickled cell separation (e.g., through filtration) or by other means changing the time treated red blood cells remain under deoxygenated condition.

In one application, the emphasis of the procedure would be on complete removal of any cells capable of sickling regardless of hemoglobin polymerization and related red blood cell sickling delays associated with such these processes. Under such approach, re-transfusion would be predominantly with hemoglobin A cells that survived in circulation from the previous red cell exchange (RCE) or single transfusion. Assuming that at the time of red cell exchange the patient has e.g., 50% of hemoglobin A cells from previous red cell exchange and 50% of patient's own hemoglobin S cells and further assuming 100% efficiency of cell separation, the amount of red blood cells (typically packed red blood cells units from storage) to be transfused would be reduced 2-fold (assuming replacement to the same hematocrit, Hb concentration or red blood cell count), as compared to standard procedure where all collected patient blood is discarded. A smaller fraction of surviving hemoglobin A cells and lower separation efficiency would result in smaller decrease in the amount of transfused red blood cells. Higher hemoglobin A fraction with high separation efficiency would result in less red blood cells being required to achieve transfusion or red cell exchange goals e.g., in terms of either or both final blood Hb concentration and blood oxygen carrying capacity and post-RCE hemoglobin S fraction in circulation.

In another application, the emphasis of the procedure would be on the reduction of the volume of transfused blood (packed red blood cells) through the reuse of all patient cells with low risk of sickling. Such cells may contain hemoglobin S, and while they would be capable of sickling under prolonged low oxygen conditions, such may be not clinically relevant if the delay in Hb polymerization of red blood cells sickling is significantly longer than the cell transit time through microvasculature. Additional consideration could be given to physiologically anticipated severity of hypoxia (low oxygen condition with more severe hypoxia corresponding to lower oxygen environment). Such severity varies between organs of the body and could also be specific to each patient due to its dependence of multiple genetic and lifestyle factors. Polymerization and sickling would be further inhibited when cells are experiencing hypoxia less than anoxia with Hb polymerization and associated red blood cell sickling further delayed or reduced in less severe hypoxia conditions. Based on the available clinical knowledge, clinician can elect to preserve maximum amount of patient's own cells that in clinician's opinion would offer low risk of sickling for the patient thus further reducing the amount of stored packed red blood cell to be transfused. Following the previous example with patient's blood containing 50% of HbAA cells and 50% of hemoglobin S containing cells, and 100% efficiency of separation, the deoxygenation conditions could be set such that only 25% of hemoglobin S cells, presumably with the highest hemoglobin S content and thus most prone to sickling, would sickle, be removed, and discarded. The remaining 75% of patient's own cells would be returned to circulation. In this scenario, only 25% of the cells would need to be replaced assuming replacement to the same hematocrit level. That would be only a quarter of the cells that would have been transfused if all patient blood would have been discarded as is done under the current procedure.

### EXAMPLES

The following are examples of infusion processes and determinations in accordance with the version of the invention shown in FIGS. 7A, 7B, and/or 7C.

### Example 1

Transfusion goal: maintain hemoglobin S percentage at a pre-defined level (e.g., at 30%) while maintaining patient total blood volume constant (significantly reduces the risks of volume overload). Note, post-procedure total hematocrit (percent of cell volume to total blood volume) can be either kept constant or changes as per clinician's preference. In some cases, increase in hematocrit may be advantageous to the patient (e.g. in case of severe anemia and compromised oxygen delivery), but it also can be detrimental to patient health e.g., as higher hematocrit leads to elevated blood viscosity and potentially higher risks of vaso-occlusion.

Step 1. Determine the percent hemoglobin S in patient blood.

Step 2. Calculate the amount of hemoglobin S containing cells to be removed and replaced with hemoglobin A containing cells from the transfused units to achieve predefined post-blood exchange final percent of hemoglobin S in patient blood.

Step 3. Calculate the volume of blood to be removed from the patient.

Option A. Volume is small (suitable for single-step phlebotomy that would not put the patient at risk due to blood loss).

Step A1. Remove the calculated volume of blood from the patient.

Step A2. Treat removed blood in accordance with step 115.

Step A3. Reintroduce treated blood back to the patient.

Step A4. Transfuse the patient with the calculated volume of packed hemoglobin A red blood cells.

Alternatively, step A1 can be replaced by an estimate of the percent hemoglobin S in patient blood based on the amount of red blood cells that would sickle as a result of complete (no or very little oxygen) blood deoxygenation and then separated in step 115. Such evaluation can be performed by simple measurement of total hemoglobin e.g., by using commercially available HemoQue systems, in the sample of blood removed from the patient as compared to the total hemoglobin in the sample of non-sickled red blood cells to be reintroduced to the patient corrected as may be necessary to the change of the sample volume.

Option B. Volume is large. (phlebotomy would be performed in several

incremental steps with blood both autologous and from the blood units being transfused back to the patients after each phlebotomy step).

Step B1. Remove the first volume of blood from the patient.

Step B2. Treat blood in accordance with step 115.

Step B3. Calculate the amount of hemoglobin A containing cells to be transfused—that is translated into the packed red blood cell volume to be transfused using unit hematocrit/hemoglobin concentration, measured or known average for units of a given type (e.g., based on storage solution, manufacturing method, etc.) equal to the amount of separated hemoglobin S containing red blood cells keeping patient blood volume and hematocrit (if desired) constant

Step B4. Reintroduce treated blood from the first volume back to the patient.

Step B5. Transfuse the patient with the calculated first volume of packed hemoglobin A red blood cells.

Step B6. Repeat the steps with the second and so on volumes of blood removed from the patient.

Option C. Options A and B with no calculations.

Step C1. Treat blood in accordance with step 115.

Step C2. (Optional) Transfuse the patient with packed hemoglobin A stored red blood cells e.g., in the amount of sickled patient red blood cells that were separated and not re-introduced into the patient.

Step C3. Estimate the size of Hemoglobin S fraction in terms of hemoglobin S containing red blood cells that are susceptible to sickling under the utilized deoxygenation level or other method.

Step C4. Repeat steps 1 through 3 until the estimated amount of hemoglobin S in patient blood reached the predefined level.

### Example 2

Method for patient-specific treatment aimed to remove hemoglobin S containing red blood cells that present maximum probability of sickling for a particular patient for e.g., patient condition, anticipate or established clinical risk, or patient lifestyle. Optionally, replacing separated (not re-introduced) sickled patient red blood cells with stored packed hemoglobin A containing red blood cells (e.g., when the volume of separated and not re-introduced red blood cells is large enough to significantly decrease patient hematocrit with the potential to create clinically undesirable anemia).

Step 1. Such level can be established based on physician's understanding of a given patient's condition, medical history, pulse oximetry measurements and other tests, based on measured changes in blood deoxygenation during exercise assessment or other approaches indication of anticipated patient-specific blood deoxygenation levels during normal activities or exercise.

Step 2. Treat blood in accordance with step 115 with maximum blood

deoxygenation corresponding to the deoxygenation level established in step 1. As a result of such limitation on the deoxygenation level, hemoglobin S containing red blood cells with lower propensity to sickling (e.g., due to elevated hemoglobin F content of with higher intracellular concentration of sickling inhibiting drug like Oxbryta) would remain un-sickled and would not be separated during the process 115 and would then be re-introduced to the patient. Only the cells with higher propensity to sickling, corresponding to the maximum blood deoxygenation level established in step 1 would sickle and then separated out and not re-introduced to the patient.

Step 3. (Optionally) Transfuse the patient with stored hemoglobin A packed red blood cells e.g., in the amount corresponding to the amount of removed sickled patient red blood cells.

Step 4. Repeat process 115 as necessary progressively removing red blood cells that sickled and optionally replacing them with stored packed hemoglobin A red blood cells.

### Example 3

Inducing a therapeutic effect by generation and re-introduction to the patient carboxy form on hemoglobin (CO-HB) in addition to the removal of patient red blood cells most susceptible to sickling.

Step 1. Remove a first volume of patient blood.

Step 2. Irradiate the blood with the wavelength and dose of irradiation selected to (a) induce blood deoxygenation to a desired level, (b) induce CO-Hb formation to a desired level.

Step 3. Separate sickled red blood cells and re-introduce non-sickled red blood cells to the patient.

Step 4. (Optionally) transfuse the patient with packed hemoglobin A containing red blood cells.

Step 5. (Optionally) Remove the second volume of blood and follow steps 2 through 4.

### Example 4

Inducing a therapeutic effect by generation and re-introduction to the patient carboxy form on hemoglobin (CO-HB) without the removal of patient red blood cells most susceptible to sickling.

Step 1. Remove a first volume of patient blood.

Step 2. Irradiate the blood with the wavelength and dose of irradiation optimized to induce maximum Co-Hb formation or alternatively, formation of desired fraction of Co-Hb.

Step 3. (Optionally) transfuse the patient with packed hemoglobin A containing red blood cells.

Step 4. (Optionally) Remove the second volume of blood and follow steps 2 through 3.

### Example 5

Using the steps of process 115 in combination with packed red blood cells storage to eliminate the delays in blood processing that may be associated with performing the process 115.

Step 1. Perform the steps as in process 1, except that the patient blood that would have been re-transfused to the patient is processed for storage instead.

Step 2. At the steps requiring re-introduction of treated patient blood processed according to step 115, transfuse the patient with autologous blood processed according to the step 115, collected and stored during the previous procedure. Non-sickled blood collected and processed for storage under current procedure would be stored until the time of the next treatment.

### Example 6

Using process 115 to create autologous packed red blood cells with reduced sickle-prone cells for future autologous transfusion (e.g., post or during surgery or other procedure or condition necessitating blood transfusion).

Step 1. Remove the first volume of blood from the patient as may be allowed by clinical blood donation guidelines.

Step 2. Use the steps of the process 115, with the separated red blood cells that would have been re-transfused to the patient being processed for storage instead.

Step 3. (Optionally) Remove the second (third, etc.) volume of blood and process it for storage.

Step 4. Transfuse the patient with autologous stored blood with low propensity for sickling as may be required.

Although the present invention has been described in considerable detail with regard to certain preferred versions thereof, other versions are possible, and alterations, permutations and equivalents of the versions shown will become apparent to those skilled in the art upon a reading of the specification and study of the drawings. For example, the cooperating components may be reversed or provided in additional or fewer number, and all directional limitations, such as up and down and the like, can be switched, reversed, or changed as long as doing so is not prohibited by the language herein with regard to a particular version of the invention. Like numerals represent like parts from figure to figure. When the same reference number has been used in multiple figures, the discussion associated with that reference number in one figure is intended to be applicable to the additional figure(s) in which it is used, so long as doing so is not prohibited by explicit language with reference to one of the figures. Also, the various features of the versions herein can be combined in various ways to provide additional versions of the present invention. Furthermore, certain terminology has been used for the purposes of descriptive clarity, and not to limit the present invention. Throughout this specification and any claims appended hereto, unless the context makes it clear otherwise, the term “comprise” and its variations such as “comprises” and “comprising” should be understood to imply the inclusion of a stated element, limitation, or step but not the exclusion of any other elements, limitations, or steps. Throughout this specification and any claims appended hereto, unless the context makes it clear otherwise, the term “consisting of” and “consisting essentially of” should be understood to imply the inclusion of a stated element, limitation, or step and the exclusion of any other elements, limitations, or steps or the exclusion of any other essential elements, limitations, or steps, respectively. Throughout the specification, any discussion of a combination of elements, limitations, or steps should be understood to include (i) each element, limitation, or step of the combination alone, (ii) each element, limitation, or step of the combination with any one or more other element, limitation, or step of the combination, (iii) an inclusion of additional elements, limitations, or steps (i.e. the combination may comprise one or more additional elements, limitations, or steps), and/or (iv) an exclusion of additional elements, limitations, or steps or an exclusion of essential additional elements, limitations, or steps (i.e. the combination may consist of or consist essentially of the disclosed combination or parts of the combination). All numerical values, unless otherwise made clear in the disclosure or prosecution, include either the exact value or approximations in the vicinity of the stated numerical values, such as for example about +/−ten percent or as would be recognized by a person or ordinary skill in the art in the disclosed context. The same is true for the use of the terms such as about, substantially, and the like. Also, for any numerical ranges given, unless otherwise made clear in the disclosure, during prosecution, or by being explicitly set forth in a claim, the ranges include either the exact range or approximations in the vicinity of the values at one or both of the ends of the range. When multiple ranges are provided, the disclosed ranges are intended to include any combinations of ends of the ranges with one another and including zero and infinity as possible ends of the ranges. Therefore, any appended or later filed claims should not be limited to the description of the preferred versions contained herein and should include all such alterations, permutations, and equivalents as fall within the true spirit and scope of the present invention.

What is claimed is:

1. A method for treating sickle cell disease, the method comprising:
removing blood from a patient with sickle cell disease;
treating the removed blood to decrease the fraction of sickle-prone cells in the removed blood; and
reintroducing the treated blood back into the patient,
wherein the treated blood that is reintroduced back into the patient has a reduced number of sickle-prone red blood cells when compared to the blood removed from the patient.

2. The method according to claim 1 wherein the treated blood that is reintroduced back into the patient has a reduced number of sickled red blood cells when compared to the blood removed from the patient.

3. The method according to claim 1 wherein the treated blood that is reintroduced back into the patient has a reduced number of sickle-prone red blood cells that had not yet sickled when the blood was removed from the patient when compared to the blood removed from the patient.

4. The method according to claim 1 wherein the step of treating the removed blood to decrease the fraction of sickle-prone cells in the removed blood comprises:
inducing sickling of at least some sickle-prone cells, and
separating the sickling-induced blood into a first portion with at least some sickled cells removed and a second portion with a higher fraction of sickled cells than in the removed blood.

5. The method according to claim 4 wherein the step of inducing sickling of at least some sickle-prone cells comprises deoxygenating hemoglobin in at least a portion of the red blood cells in the removed blood.

6. The method according to claim 4 wherein the step of inducing sickling of at least some sickle-prone cells comprises deoxygenating hemoglobin in at least a portion of the red blood cells in the removed blood by enzymatic oxygen consumption.

7. The method according to claim 4 wherein the step of inducing sickling of at least some sickle-prone cells comprises deoxygenating hemoglobin in at least a portion of the red blood cells in the removed blood by photochemical modification.

8. The method according to claim 4 wherein the step of inducing sickling of at least some sickle-prone cells comprises deoxygenating hemoglobin in at least a portion of the red blood cells in the removed blood by gas exchange.

9. The method according to claim 4 wherein the step of inducing sickling of at least some sickle-prone cells comprises deoxygenating hemoglobin in at least a portion of the red blood cells in the removed blood by chemically-induced oxygen scrubbing.

10. The method according to claim 4 wherein the step of inducing sickling of at least some sickle-prone cells comprises deoxygenating hemoglobin in at least a portion of the red blood cells in the removed blood by natural deoxygenation.

11. The method according to claim 4 wherein the step of separating the sickling-induced blood comprises filtering the sickling-induced blood.

12. The method according to claim 4 wherein the step of separating the sickling-induced blood comprises passing the sickling-induced blood through one or more filters having a pore size of from about from about 2 microns to about 10 microns.

13. The method according to claim 4 wherein the step of separating the sickling-induced blood comprises density separation.

14. The method according to claim 1 further comprising introducing stored blood into the patient.

15. The method according to claim 14 wherein the stored blood comprises donor red blood cell concentrate.

16. The method according to claim 1 further comprising introducing stored blood into the patient in a volume selected in relation to the volume lost from the treatment of the removed blood.

17. The method according to claim 1 wherein the treated blood is stored before being reintroduced back into the patient.

18. A method for treating sickle cell disease, the method comprising:
removing blood from a patient with sickle cell disease;
inducing sickling of at least some sickle-prone cells in the removed blood;
removing at least some sickled cells from the sickling-induced blood; and
reintroducing the blood with the removed sickled cells back into the patient.

19. The method according to claim 18 wherein the step of inducing sickling of at least sickle-prone cells comprises deoxygenating hemoglobin in at least a portion of the red blood cells in the removed blood by one or more of enzymatic oxygen consumption, photochemical modification, gas exchange, chemically-induced oxygen scrubbing, and natural deoxygenation.

20. The method according to claim 18 wherein the step of removing at least some sickled cells from the sickling-induced blood comprises one or more of filtration and density separation.

21. The method according to claim 18 further comprising introducing stored blood into the patient in a volume selected in relation to the volume lost from the removing at least some sickled cells from the sickling-induced blood.

22. The method according to claim 18 wherein the blood with the removed sickled cells is stored before being reintroduced back into the patient.

23. A sickle cell disease treatment system comprising:
a line adapted to receive blood from a patient;
a blood treatment unit in communication with the line adapted to receive blood from a patient, the blood treatment unit comprising:
a sickling inducement unit adapted to receive blood from the patient and induce sickling of sickle-prone cells in the blood from the patient; and
a sickled cell separation unit adapted to remove sickled cells from the blood received from the sickling inducement unit; and
a line adapted to reintroduce treated blood from the blood treatment unit back into the patient,
wherein the reintroduced blood has a reduced number of sickle-prone red blood cells when compared to the blood removed from the patient.
